SEQUENCE LISTING (14 sequences)

SEQ ID NO: 1 - DNA, 32 bp, Artificial Sequence
  primer
aatggtacccaacatgaacaccatcccggcac

SEQ ID NO: 2 - DNA, 34 bp, Artificial Sequence
  primer
aatgcggccgctcaagatatttgcgttgcaattc

SEQ ID NO: 3 - DNA, 18 bp, Artificial Sequence
  primer
tggtcggcgcccaattcc

SEQ ID NO: 4 - DNA, 26 bp, Artificial Sequence
  primer
agagcctagaggtactctgtgggcgg

SEQ ID NO: 5 - DNA, 33 bp, Artificial Sequence
  primer
ataaggtaccaaaatggcttccaaggtgtacga

SEQ ID NO: 6 - DNA, 32 bp, Artificial Sequence
  primer
ataagcggccgcttactgctcgttcttcagca

SEQ ID NO: 7 - DNA, 33 bp, Artificial Sequence
  primer
gcttcgtctacggagcgacaattcaattcaaac

SEQ ID NO: 8 - DNA, 35 bp, Artificial Sequence
  primer
gcagtagcctcatcatcactagatggcatttcttc

SEQ ID NO: 9 - DNA, 22 bp, Artificial Sequence
  primer
tcaaacaagcaaagtgaacacg

SEQ ID NO: 10 - DNA, 24 bp, Artificial Sequence
  primer
ccattcatcagttccataggttgg

SEQ ID NO: 11 - DNA, 37 bp, Artificial Sequence
  primer
ttatgtacaagtacccatacgatgttcctgactatgc

SEQ ID NO: 12 - DNA, 41 bp, Artificial Sequence
  primer
taagaattcttaagcgtaatctggaacgtcatatggatagg

SEQ ID NO: 13 - DNA, 20 bp, Artificial Sequence
  primer
gctgcagtttgtggcgagtg

SEQ ID NO: 14 - DNA, 20 bp, Artificial Sequence
  primer
aaccgatcccgctccatttc

(19) United States
(12) Patent Application Publication
(10) Pub. No.: US 2011/0277050 A1
(43) Pub. Date: Nov. 10, 2011

(54) SYSTEM AND METHODS TO CONTROL TRANSGENE EXPRESSION

(75) Inventors: Liqun Luo, Palo Alto, CA (US); Christopher Potter, Baltimore, MD (US); Bosiljka Tasic, Palo Alto, CA (US)

(73) Assignee: The Board of Trustees of the Leland Stanford Junior University, Palo Alto, CA (US)

(21) Appl. No.: 13/046,035

(22) Filed: Mar. 11, 2011

Related U.S. Application Data
(60) Provisional application No. 61/313,680, filed on Mar. 12, 2010.

(51) Int. Cl. A01K 67/02; C12Q 1/68; C12N 15/87

(52) U.S. Cl. 800/22; 435/465; 435/6.18

(57) ABSTRACT

Embodiments of the present invention describe a novel and versatile inducible binary expression system (the ‘Q system’) and methods for controlling transgene expression in vitro and in vivo, for lineage tracing, for genetic mosaic analysis and for determining gene function.

### RELATED APPLICATION

This application claims priority and other benefits from U.S. Provisional Patent Application Ser. No. 61/313,680, filed Mar. 12, 2010, entitled “System and methods to control transgene expression”. Its entire content is specifically incorporated herein by reference.

### STATEMENT OF GOVERNMENTAL SUPPORT

This invention was made with government support under Grant No. DC005982 awarded by the National Institutes of Health. The Government has certain rights in this invention.

### TECHNICAL FIELD OF THE INVENTION

The present invention relates to the field of transgene expression systems. In particular, the present invention describes an inducible binary expression system and methods to control transgene expression in cells and animals.

### BACKGROUND

The ability to introduce engineered transgenes with regulated expression into organisms has revolutionized biology. A popular strategy for regulating expression of an effector transgene is to use a binary expression system. In this strategy, one transgene contains a specific promoter driving an exogenous transcription factor, while the other transgene uses the promoter activated only by that transcription factor to drive the effector gene. As a result, the effector gene is controlled exclusively by the chosen transcription factor, and the expression pattern of the effector transgene corresponds to the expression pattern of the exogenous transcription factor.

A number of binary expression systems have been established in genetic model organisms over the years, including tetracycline-regulable tTA/Tetracycline Response Element (TRE) and GAL4/Upstream Activating Sequence (UAS) binary expression systems. Despite the many advances that such systems have afforded for studies of biological systems, improvement of precision of expression as well as increased versatility and controllability are still needed to fully realize the potential of expression systems.

### SUMMARY

Embodiments of the present invention describe a novel and versatile inducible binary expression system (the ‘Q system’) and methods to control transgene expression in vitro and in vivo. A further embodiment of the present invention describes a novel and versatile inducible binary expression system and methods for lineage tracing. Yet another embodiment of the present invention describes a novel and versatile inducible binary expression system and methods for genetic mosaic analysis. A further embodiment of the present invention describes a novel and versatile inducible binary expression system and methods for determining gene function.

The above summary is not intended to include all features and aspects of the present invention nor does it imply that the invention must include all features and aspects discussed in this summary.

### INCORPORATION BY REFERENCE

All publications mentioned in this specification are herein incorporated by reference to the same extent as if each individual publication or patent application was specifically and individually indicated to be incorporated by reference.

### DRAWINGS

The accompanying drawings illustrate embodiments of the invention and, together with the description, serve to explain the invention. These drawings are offered by way of illustration and not by way of limitation; it is emphasized that the various features of the drawings may not be to scale.

FIG. 1 illustrates the characterization of the Q-system in Drosophila and Mammalian Cells, in accordance with embodiments of the present invention. (A) Schematic of the Q repressible binary expression system. In the absence of the transcription factor, QF, the QF-responsive transgene, QUAS-X, does not express X (top). When QF and QUAS-X transgenes are present in the same cell where QF is expressed (promoter P1 is active), QF binds to QUAS and activates expression of gene X (middle). When QS, QF and QUAS-X transgenes are present in the same cell, and both P1 and P2 promoters are active, QS represses QF and X is not expressed (bottom).

(B) Characterization of the Q-system in transiently transfected Drosophila S2 cells. Relative luciferase activity (normalized as described in experimental procedures, infra) is plotted on a logarithmic scale on the y-axis, with QUAS-luc2 alone set to 1. Error bars are SEM. Plasmids used for transfections are noted below the x-axis. QUAS, pQUAS-luc2 reporter; QF, pAC-QF; QS, pAC-QS; UAS, pUAS-luc2 reporter; G4, pAC-GAL4; G80, pAC-GAL80; x3 and x5, 3 and 5-fold molar excess of QS over QF or GAL80 over GAL4.
(C) Characterization of the Q-system in transiently transfected human HeLa cells. Explanations and abbreviations as in (B) except: QF, pCMV-QF; QS, pCMV-QS; G4, pCMV-GAL4; G80, pCMV-GAL80.

FIG. 2 shows the effect of quinic acid (QA) on transiently transfected Drosophila and mammalian cells, in accordance with embodiments of the present invention. (A) The effect of quinic acid on transiently transfected Drosophila S2 cells. Relative luciferase activity (normalized as described in experimental procedures, infra) is plotted on a logarithmic scale on the y-axis, with QUAS-luc2 alone set to 1. Quinic acid (QA) was added where indicated to the final concentration of: QA1=12.5 μg/ml; QA2=250 μg/ml; QA3=5 mg/ml. All data are presented as average +SEM. Statistical significance was evaluated using Student's t-test; ns, not significant; *, p<0.05; ***, p<0.001. QA, at the highest concentration tested, results in ˜23-fold re-activation of QUAS, compared to the control sample with QUAS, QF and 5xQS. At this high concentration, QA has no effect on QF activation of QUAS or Gal4 activation of UAS, and it has a small but significant effect on GAL80 repression of GAL4: it derepresses GAL4 by ˜1.5-fold. Plasmids used for transfections are noted below the x-axis. QUAS, QUAS-luc2 reporter; QF, pAC-QF; QS, pAC-QS; x5, five-fold molar excess of QS over QF. (B) The effect of quinic acid on transiently transfected human HeLa cells. The data are presented, and statistical significance was evaluated, as in (A). QA has no effect on QF-dependent transcription of QUAS, but in the presence of QF and 5xQS, it further significantly suppresses QUAS activation by ˜13-fold at the two higher QA concentrations tested. Explanations and abbreviations as in (A) except: QF, pCMV-QF; QS, pCMV-QS.

FIG. 3 illustrates exemplary in-vivo characterization of the Q system in flies, in accordance with embodiments of the present invention. (A) Representative confocal projections of whole mount Drosophila brains immunostained for a general neurophil marker (monoclonal antibody nc82) in magenta, and for mCD8 in green. Genotypes are indicated at the bottom. A3 is a higher magnification image centered at the antennal lobe (AL; outlined). QF is driven by the GH146 enhancer that labels a large subset of olfactory projection neurons (PNs). PN cell bodies (arrowheads in A3) are located in anterodorsal, lateral or ventral clusters around the AL. PNs project dendrites into the AL, and axons to the mushroom body calyx (MB) and the lateral horn (LH) outlined. The green channel for A1 and A4 was imaged under the same gain, which is 15% higher than for the images shown in A2 and A3. (B) Representative confocal projections of whole mount Drosophila brains immunostained for a general neuropil marker N-cadherin in blue, and for HA in red. The genotypes are indicated at the bottom. B3 is a higher magnification image centered at the AL (outlined). Arrowheads denote PN cell bodies. The red channel for B1 and B4 was imaged under the same gain, which is 15% higher than for the images shown in B2 and B3. The red staining in B4 is due to the DsRed transgenic marker associated with the GH146-QF transgene vector. (C) Fluorescence images of three adult flies with genotypes as indicated. (D) Fluorescence images of adult flies with genotypes indicated on top. Numbers on the bottom indicate the amount of quinic acid (dissolved in 300 μl of water) added to the surface of ˜10 ml fly food, on which these flies developed. (E) Fluorescence images of adult flies showing time course of derepression of QS by quinic acid. The adult flies of the genotype listed on top were moved from vials with regular food to vials containing 75 mg quinic acid and imaged after the time interval shown on the bottom. Scale bars: 50 μm for A1,2,4 and B1,2,4; 20 μm for A3 and B3.

FIG. 4 shows additional characterization of the Q System in vivo, in accordance with embodiments of the present invention. (A) Comparison of basal expression levels for UAS-, lexAO- and QUAS-reporter transgenes in the adult Drosophila brain. All images show maximum z-projections of confocal stacks. _{A1}-_{A10} show anti-GFP antibody staining of adult brains of: (_{A5}) a whit^{e1118} control fly, (_{A6}) a commonly used UAS-mCD8-GFP fly (Lee and Luo, 1999), (_{A1}-_{A4}) four independent insertions of lexAO-CD2-GFP (Lai and Lee, 2006), (_{A7}-_{A10}) four independent insertions of QUAS-mCD8-GFP (embodiments of the present invention). _{A11}-_{A14} show anti-HA staining of adult brains for four independent insertions of QUAS-mtdT-HA (embodiments of the present invention). All brains that were stained against GFP (A₁-A₁₀) were processed using identical staining and imaging conditions, which were set such that the anti-GFP signal in control animals (GH146-GAL4+UAS-mCD8-GFP) was fully saturated (not shown). All brains that were stained against HA (A₁₁-A₁₄) were processed using identical conditions. Imaging conditions were set such that the anti-HA signal in control animals (GH146QF+QUAS-mtdT-HA) was fully saturated (not shown). All brains were heterozygous for the driver and reporter transgenes. Line numbers or genotypes are indicated on the bottom of each image. Scale bar=50 μm. (B) Expression of QF enhancer trap lines and their suppression by ubiquitous expression of QS. Fluorescence images of adult flies of QF enhancer traps (ET) lines expressing the same QUAS-mtdT-HA reporter are shown. ET line numbers are indicated in the first panel for each set of four panels. All images were taken at the same exposure (400 ms), except for ET6-QF, which was taken at a higher exposure. Inset panels for ET6-QF show the same images at the 400 ms exposure. Inset panels for ET14-QF and ET12-QF are higher magnifications of the abdomen showing that each bristle socket is labeled. The animals are ordered from lowest expressing line (ET6-QF) to highest expressing line (ET49-QF) from top to bottom and continuing to the right column. Expression of each ET line can be suppressed by tubP-QS, although occasionally a few cells remained labeled with ET49-QF (not shown). White outlines mark the positions of the adult flies and magnified heads in the presence of tubP-QS. These QF enhancer trap lines exhibited no gross defects caused by QF expression. Line ET10-QF exhibited a crumpled wing, but this is likely due to a mutational insertion—other QF enhancer trap lines expressing more highly in similar tissues appeared normal (e.g. ET40-QF, ET9-QF, ET49-QF).

FIG. 5 illustrates examples of the expression of QF enhancer trap lines in adult fly brain, in accordance with embodiments of the present invention. (A) Representative limited z-projections of confocal stacks of whole mount Drosophila brains containing a QF enhancer trap (line number indicated on top right) and QUAS-mCD8-GFP. The brains were immunostained for a general neuropil marker (monoclonal antibody nc82) in magenta, and for mCD8 in green. The QF enhancer trap line number is indicated within each panel. The lines are organized as in FIG. 4B. All lines except ET40-QF express in trachea to varying degrees. Notable expression patterns are: ET6-QF, ET10-QF, ET14-QF, and ET12-QF—mostly trachea with few neurons; ET31-QF—ensheathing glia (see FIG. 5C); ET11-QF—AL, CC, and MB; ET8-QF—general expression in brain tissues, especially in the AL, CC, MB, OL and SOG; ET13-QF—expression throughout the brain; ET40-QF—AL, MB, OL and SOG; ET9-QF and ET49-QF—strong expression throughout the brain including AL, CC, MB and SOG. Full confocal stack files available upon request. Abbreviations: AL, antennal lobe; CC, central complex; MB, mushroom bodies; OL, optic lobe; SOG, subesophageal ganglion. Scale bars: 50 μm. (B) GH146-QF labels olfactory PNs. The PN nuclei are labeled by GH146-QF driven QUAS-nuclear-LacZ (nucLacZ, anti-LacZ in green) and PN morphology is outlined by QUAS-mtdT-HA (anti-HA in red) transgenes. Note that all red neurons contain green nuclei. There are some scattered green-only cells (LacZ positive, mtdT-HA negative). As nuclear-LacZ is likely a more stable marker than mtdT-HA (our unpublished observations), the green-only cells might represent those that expressed GH146 transiently during development. Dotted lines represent the antennal lobes, which contain PN dendrites. Scale bars: 20 μm.

(C) ET31-QF labels ensheathing glia. (TOP) Limited z-projection confocal images of ET31-QF driving mCD8-GFP expression (left panel). The labeled cells ensheath neuronal tissues, such as the glomeruli of the antennal lobe (anterior section, middle panel) and mushroom body lobes (posterior section, right panel). (MIDDLE) Full z-projection confocal image of ET31-QF driving nuclear LacZ expression. The brain was stained against lacZ (green) and against the glial marker Repo (red); cells expressing both markers appear yellow (left panel). Limited z-projection confocal images of anterior (middle panel) and posterior (right panel) sections show that many Repo-positive cells are labeled by ET31-QF. (BOTTOM) Full z-projection confocal image of ET31-QF driving nuclear LacZ expression (green), co-stained with an antibody against the neuronal marker ELAV (red); cells expressing both markers appear yellow (left panel). Limited z-projection confocal images of anterior (middle panel) and posterior (right panel) sections show that ET31-QF rarely labels ELAV-positive cells. Cells labeled by ET31-QF that are both Repo-negative and ELAV-negative most likely represent tracheal cells. The white boxes in the left panels show the brain regions that are magnified in the middle and right panels. Scale bars: 50 μm for left panels; 20 μm for middle and right panels. (D-E) Enhancer trap lines ET9-QF and ET49-QF label many neurons. Full z-projections of confocal images of Drosophila brains containing ET9-QF or ET49-QF driving nuclear LacZ expression. The brains were stained against lacZ (green) and against the neuronal marker ELAV (red) (left panels). Limited z-projection confocal images of anterior (middle panels) and posterior (right panels) sections show that many neurons are labeled by these ET lines. Mushroom body neurons are outlined in images on the right. Scale bars: 50 μm for left panels; 20 μm for middle and right panels.

FIG. 6 illustrates examples of Q-MARCM and Coupled MARCM, in accordance with embodiments of the present invention. (A) Scheme for Q-MARCM. FLP/FRT mediated mitotic recombination in G2 phase of the cell cycle (dotted cross) followed by chromosome segregation as shown causes the top progeny to lose both copies of tubP-QS, and thus becomes capable of expressing the GFP marker (G) activated by QF. It also becomes homozygous for the mutation (*). QF and QUAS reporter transgenes can be located on any other chromosome arm. P1, promoter 1. tubP, tubulin promoter. Centromeres are represented as circles on chromosome arms. (B) Q-MARCM clones of olfactory PNs visualized by GH146-QF driven QUAS-mCD8-GFP. (B1-B2) Confocal images of an anterodorsal neuroblast clone showing cell bodies of PNs (arrowhead), their dendritic projections in the antennal lobe (arrows) and axonal projections in the MB and LH (outlined). (B3-B4) Confocal images of a single cell clone showing the cell body of a DL1 PN (arrowhead), its dendritic projection into the DL1 glomerulus (arrow) of the antennal lobe and its axonal projection in the MB and LH (outlined). (C) Scheme for coupled MARCM. The tubP-GAL80 and tubP-QS transgenes are distal to the same FRT on homologous chromosomes. Mitotic recombination followed by specific chromosome segregation produces two distinct progeny devoid of QS or GAL80 transgenes, respectively, and therefore capable of expressing red (R) or green (G) fluorescent proteins, respectively. QF and GAL4 transgenes (not diagramed), as well as QUAS and UAS transgenes, can be located on any other chromosome arm. ‘*’ and ‘x’ designate two independent mutations that can be rendered homozygous in sister progeny. (D) A coupled MARCM clone of photoreceptors, showing clusters of cell bodies (arrowheads) in the eye imaginal disc and their axonal projections (arrows) to the brain. The green clone was labeled by tubP-GAL4 driven UAS-mCD8-GFP; the red clone was labeled by ET40-QF driven QUAS-mtdT-HA. Blue, DAPI staining for nuclei. Image is a z-projection of a confocal stack. Scale bars: 20 μm.

FIG. 7 shows the lack of cross-activation and cross-repression of the Q and GAL4 systems in vivo, and a schematic of independent double MARCM, in accordance with embodiments of the present invention. (A) QF and GAL4 systems do not cross-activate in vivo. (First to Third row) Representative confocal projections of whole mount Drosophila brains immunostained for a general neurophil marker (monoclonal antibody nc82) in magenta, and for mCD8 in green. The genotypes are represented by the schematics in the left panels. Higher magnification images centered at the antennal lobe are shown in the right panels. Staining and imaging conditions were similar to those of FIG. 3A. Scale bars: 50 μm for middle panels; 20 μm for right panels. (First row) GH146-QF expression does not activate a UAS-mCD8-GFP reporter. (Second row) GH146-GAL4 expression does not activate a QUAS-mCD8-GFP reporter. (Third row) GH146-QF expression does not activate UAS-FLP since mCD8-GFP was not expressed from the QUAS>stop>mCD8-GFP reporter. >, FRT. (Fourth and Fifth row) Representative confocal projections of wing imaginal discs immunostained for mCD8 in green and nuclei using DAPI in blue. The genotypes are represented by the schematics in the left panel (“!” represents transcriptional stop; yellow triangles represent FRT sites). Staining and imaging conditions were similar to those of FIG. 10 and FIG. 11C. Scale bars: 50 μm.

(Fourth row) QF expression throughout the wing imaginal disc (driven by ET40-QF) does not activate UAS-FLP since mCD8-GFP was not expressed from the QUAS>stop>mCD8-GFP reporter. 50 wing imaginal discs were examined. Eye imaginal discs (n=30) also showed no reporter expression (not shown). >, FRT. (Fifth row) GAL4 expression throughout the wing imaginal disc (driven by tubP-GAL4) does not activate QUAS-FLP since mCD8-GFP was not expressed from the UAS>stop>mCD8-GFP (right panel) reporter. 20 wing imaginal discs were examined. Eye imaginal discs (n=6) also showed no reporter expression (not shown). >, FRT.

(B) QF and GAL4 systems do not cross-repress in vivo. Representative confocal projections of whole mount Drosophila brains immunostained for a general neuropil marker (monoclonal antibody nc82) in magenta, and for mCD8 in green. The genotypes are represented by the schematics on the left. Higher magnification images centered at the antennal lobe are shown on the right. Comparison of first and second rows indicates that GH146-QF expression, as reported by QUAS-mCD8-GFP, is not affected by ubiquitous expression of GAL80. Comparison of third and fourth rows indicate that GH146-GAL4 expression, as reported by UAS-mCD8-GFP, is not affected by ubiquitous expression of QS. All experimental conditions (staining protocol, antibody concentrations, number of brains per staining, imaging conditions) were identical. Scale bars: 50 μm for middle panels; 20 μm for right panels.

(C) Schematic of independent double MARCM. tubP-GAL80 and tubP-QS are placed on two different chromosome arms. Two types of mitotic recombination events (1 and 2) are independent of each other and result in differently labeled progeny. Cells homozygous for a single mutation (x or *) are singly colored green or red, respectively, while cells homozygous for both mutations appear yellow. Additional UAS or QUAS transgenes can be included into the scheme, so that progeny containing active GAL4 or QF can express additional markers or effector genes. Centromeres are represented as circles on the chromosomes. This type of manipulation will be useful for studying gene function in cell-cell interactions and in comparing phenotypes of single and double mutants in the same animal.

FIG. 8 shows a lineage analysis using coupled MARCM, in accordance with embodiments of the present invention. (A) General scheme for neuroblast division in the insect CNS. Nb, neuroblast; GMC, ganglion mother cell; N, postmitotic neuron.(B) Three types of MARCM clones predicted from the general scheme. M, mitotic recombination.(C) Three models to account for the lack of two cell clones in GH146-labeled MARCM. (C1) Each neuroblast division directly produces a postmitotic GH146-positive PN without a GMC intermediate. (C2) Each GMC division produces a GH146-positive PN and a GH146-negative cell. (C3) Each GMC division produces a GH146-positive PN and a sibling cell that dies. For models II and III, simulations of coupled MARCM results are shown for mitotic recombination that occurs either in the neuroblast or in the GMC. (D) Tabulation of coupled MARCM results. Superscripts next to the numbers correspond to the images shown in (E) as examples. (E) Examples of coupled MARCM that contradict models I and II, but can be accounted for by model III (bottom). (E1-E2) A single QF-(E1) or GAL4-(E2) labeled PN in the absence of labeled siblings. These events contradict model I (C1). In both examples, the additional green staining in the antennal lobe belongs to tubP-GAL4 labeled axons from olfactory receptor neurons. (E3) A single tubP-GAL4 labeled sibling (green) of a GH146-QF labeled neuroblast clone (red). This observation contradicts model II (C2). (E4) An occasional QF-labeled neuroblast clone with no tubP-GAL4 labeled siblings. All images are z-projections of confocal stacks; green, anti-CD8 staining for UAS-mCD8-GFP; red, anti-HA staining for QUAS-mtdT-HA; blue, neurophil markers. Arrowheads, PN cell bodies; arrows, dendritic innervation in the antennal lobe (outlined). Scale bars: 20 μm.

FIG. 9 illustrates a schematic for coupled MARCM for PN lineage analysis, in accordance with embodiments of the present invention. Mitotic recombination followed by specific chromosome segregation produces two distinct progeny. One progeny is devoid of the QS transgene and is therefore capable of expressing a red fluorescent protein (R, QUAS-mtdT-HA) via GH146-QF in PNs. The other progeny is devoid of the GAL80 transgene and is therefore capable of expressing a green fluorescent protein (G, UAS-mCD8-GFP) via tubP-GAL4 in any cell that is a sibling of the red cell above. FLP expression during development is mediated by a 1 h heat-shock induction of a hs-FLP transgene (not diagrammed).

FIG. 10 illustrates examples of coupled MARCM for clonal analysis of mutant phenotypes, in accordance with embodiments of the present invention. (A) Schematic for coupled MARCM labeling of dividing cells during imaginal disc development.(B) A control coupled MARCM clone. Both GAL4- and QF-labeled siblings are wild type. Genotype: hsFLP, QUAS-mtdT-HA, UAS-mCD8-GFP(X); ET40-QF, QUAS-mtdT-HA/+ (II); tubP-GAL4, 82BFRT, tubP-GAL80/82BFRT, tubP-QS (III). (C) A coupled MARCM clone where GAL4-labeled sibling (green) is wild type, while QF-labeled sibling (red) is homozygous mutant for Tsc1. Genotype: hsFLP, QUAS-mtdT-HA, UAS-mCD8-GFP(X); ET40-QF, QUAS-mtdT-HA/+(II); tubP-GAL4, 82BFRT, tubP-GAL80, Tsc1Q600X/82BFRT, tubP-QS (III). Green, anti-CD8; Red, anti-HA; Blue, anti-fibrillarin (labels nucleoli). Scale bars: 20 μm. (D-F) Quantification of clone area, cell number and cell size for experiments in B and C. n=30 for WT vs. WT; n=21 for WT vs. Tsc1. Error bars are ±SEM. ***, p<0.001.

FIG. 11 shows additional characterization of the effects of QF, GAL4, or QF+GAL4 expression on imaginal disc differentiation, in accordance with embodiments of the present invention. (A) Eye disc differentiation is not affected by QF expression. Third instar eye imaginal discs were stained for nuclei (DAPI), HA (from QUAS-mtdT-HA), 24B10 (photoreceptor specific marker), and ELAV (neuronal differentiation marker) to monitor normal differentiation. Marker expression appears indistinguishable in eye imaginal discs that express QF throughout eye disc development (ET40-QF, rows three and four) and in control wild-type imaginal discs (Canton S, rows one and two). Scale bars: 50 μm for rows 1 and 3; 20 μm for rows 2 and 4. Rows 2 and 4 show higher magnification images of eye discs from Rows 1 and 3. (B) Adult eye section of ET40-QF animals, which expressed QF throughout eye disc development, exhibits no defects. The dashed line labels the dorsal/ventral equatorial border.

(C) Expression of QF and GAL4 together in wing imaginal discs does not affect expression levels of QF and GAL4 reporters. Third instar eye imaginal discs of the genotypes listed on the left were stained for nuclei (DAPI), mCD8 (from UAS-mCD8-GFP), and HA (from QUAS-mtdT-HA). Expression of QF does not affect the levels of GAL4-mediated reporter expression (compare anti-mCD8 signal in top row and third row), and expression of GAL4 did not affect QF-mediated reporter expression (compare anti-HA signal in second and third rows). To limit experimental variability during immunostaining, 15 wings discs for each of the three genotypes were stained together in the same tube. Scale bars: 50 μm. (D) Wing disc expression of QF, or coexpression of QF and GAL4, does not affect adult wing morphogenesis. Light microscope images of whole adult wings and magnified images of the anterior wing regions are shown for the genotypes listed on top. The development and morphogenesis of the adult wing (as monitored by vein pattern, bristle orientation and wing size) is unaffected by expression of QF, GAL4, or QF+GAL4. For each genotype, we examined at least 20 wings and all wings appear normal with the exception that one out of 46 wings in the QF+GAL4 condition exhibited an incomplete L3-4 cross vein. Wing sizes were calculated in ImageJ by measuring the wing area (excluding the wing hinge). No significant differences in wing size were found.

FIG. 12 shows intersectional methods to refine transgene expression, in accordance with embodiments of the present invention. (A1) Schematic showing two partially overlapping cell populations: one expressing an acj6-GAL4-driven green marker (within the left rectangle), and the other expressing a GH146-QF-driven red marker (within the right rectangle). Cells in the center express both GAL4 and QF and appear yellow. (A2-A4) Single confocal sections (A2, A4) or a z-projection (A3) of the adult antennal lobe (A2-A3) or mushroom body calyx (A4) from flies with the genotype shown in A1. Green, red and yellow cells in A2 represent PNs that express acj6-GAL4 only, GH146-QF only, or both, respectively. Their dendrites form green, yellow and red glomeruli (A2). Their axons form green, red, and yellow terminal boutons in the mushroom body (A4). (A3) is the z-projection of the red channel for A2; the oval highlights cell bodies of anterodorsal PNs. Green: anti-CD8 staining for UAS-mCD8-GFP; Red: anti-HA staining for QUAS-mtdT-HA. Blue: neuropil marker. (B1) Schematic for “QF NOT GAL4” for acj6-GAL4 and GH146-QF. UAS-QS is added to A1, resulting in the repression of QF activity in cells that express both QF and GAL4 (center). QF reporter expression is thus subtracted from the overlapping population of cells. (B2-B4) Equivalent samples as A2-A4, except with UAS-QS added. Compared to A3, anterodorsal PNs no longer express QUAS-mtdT-HA (dotted oval in B3). There are no yellow cells and glomeruli in the antennal lobe (B2), or yellow terminal boutons in the mushroom body (B4). Note: In the experiments shown in A and B, to clearly visualize only non-ORN processes in the antennal lobe, antennae and maxillary palps were removed 10 days prior to staining, causing all Acj6-expressing ORN axons to degenerate. (C1) Schematic for “QF AND GAL4” for acj6-GAL4 and GH146-QF. GAL4 driven FLP results in the removal of a transcriptional stop (!) from a QUAS reporter (within the left rectangle), but the reporter can only be expressed in cells where QF is expressed (within the right rectangle). Thus, only the cells in the overlap (center) express the reporter. (C2) Confocal stack of a whole mount central brain showing reporter (mCD8-GFP) expression from acj6-GAL4, which labels many types of neurons including most ORNs, olfactory PNs and optic lobe neurons. (C3-C4) The AND gate between GH146-QF and acj6-GAL4 (genotype as in C1) limits mCD8-GFP expression to a cluster of anterodorsal PNs and a single lateral neuron (arrowhead in C4). Arrow in C3, axons of anterodorsal PNs.

(D1) Schematic for “QF AND GAL4” similar to C1, but for NP21-GAL4 and GH146-QF.(D2) Confocal stack of whole mount central brain showing reporter (mCD8-GFP) expression from NP21-GAL4. (D3-D4) The AND gate between GH146-QF and NP21-GAL4 limits reporter expression to a few classes of PNs that project to several glomeruli including DA1 (arrow in D4) and to neurons that project to the ellipsoid body (arrow in D3). (E1) Schematic for an alternative approach to “GAL4 AND QF” for NP21-GAL4 and GH146-QF. Here, FLP is driven by QF, and the reporter is driven by GAL4.(E2) High magnification of NP21-GAL4 expression pattern centered at the antennal lobe. In the adult, only one class of lateral PNs projecting to the DA1 glomerulus (arrow) is evident. (E3-E4) This AND gate between GH146-QF and NP21-GAL4 limits expression to a single class of lateral PNs that project to the DA1 glomerulus (arrow in E4). Occasional expression is also found in a few cells in the anterior lateral region of the brain. Genotypes: (A) acj6-GAL4, GH146-QF, UAS-mCD8-GFP, QUAS-mtdT-HA; (B) acj6-GAL4, GH146-QF, UAS-mCD8-GFP, QUAS-mtdT-HA, UAS-QS; (C2) acj6-GAL4, UAS-mCD8-GFP; (C3, C4) acj6-GAL4, GH146-QF, UAS-FLP, QUAS>stop>mCD8-GFP; (D2, E2) NP21-GAL4, UAS-mCD8-GFP; (D3, D4) NP21-GAL4, GH146-QF, UAS-FLP, QUAS>stop>mCD8-GFP; (E3, E4) NP21-GAL4, GH146-QF, UAS>stop>mCD8-GFP, QUAS-FLP; “>”, FRT site. Scale bars: 20 μm.

FIG. 13 shows reporter expression patterns derived from logic gates of QF and GAL4 expression patterns, in accordance with embodiments of the present invention. Grey squares represent the original expression pattern of GAL4 or QF (top line), or the reporter (R) expression resulting from the listed logic gate. The transgenes required for the intersectional logic gates are listed in the right column. Not listed here are four remaining logic gates (A, B, FALSE, TRUE), which do not create new expression patterns. Transgenes not yet constructed are italicized. Although there are multiple ways to construct each logic gate, we show only one for each except for the AND gate, for which we have experimentally demonstrated two alternative methods. ‘>’ represents an FRT site.

FIG. 14 illustrates examples of defining PNs responsible for olfactory attraction using intersectional methods, in accordance with embodiments of the present invention. (A) Schematic of the olfactory trap assay. O,1% ethyl acetate in mineral oil; C, control (mineral oil alone). A performance index (PI) is used to measure olfactory attraction. (B) Performance index plots of flies of listed genotypes. Error bars are ±SEM.

### DEFINITIONS

The practice of the present invention may employ conventional techniques of chemistry, molecular biology, recombinant DNA, microbiology, cell biology, immunology, genetics, and biochemistry, which are within the capabilities of a person of ordinary skill in the art. Such techniques are fully explained in the literature. For definitions, terms of art and standard methods known in the art, see, for example, Sambrook and Russell ‘Molecular Cloning: A Laboratory Manual’, Cold Spring Harbor Laboratory Press (2001); ‘Current Protocols in Molecular Biology’, John Wiley & Sons (2007); William Paul ‘Fundamental Immunology’, Lippincott Williams & Wilkins (1999); M. J. Gait ‘Oligonucleotide Synthesis: A Practical Approach’, Oxford University Press (1984); R. Ian Freshney “Culture of Animal Cells: A Manual of Basic Technique’, Wiley-Liss (2000); ‘Current Protocols in Microbiology’, John Wiley & Sons (2007); ‘Current Protocols in Cell Biology’, John Wiley & Sons (2007); Wilson & Walker ‘Principles and Techniques of Practical Biochemistry’, Cambridge University Press (2000); Roe, Crabtree, & Kahn ‘DNA Isolation and Sequencing: Essential Techniques’, John Wiley & Sons (1996); D. Lilley & Dahlberg ‘Methods of Enzymology: DNA Structure Part A: Synthesis and Physical Analysis of DNA Methods in Enzymology’, Academic Press (1992); Harlow & Lane ‘Using Antibodies: A Laboratory Manual: Portable Protocol No. I’, Cold Spring Harbor Laboratory Press (1999); Harlow & Lane ‘Antibodies: A Laboratory Manual’, Cold Spring Harbor Laboratory Press (1988); Roskams & Rodgers ‘Lab Ref: A Handbook of Recipes, Reagents, and Other Reference Tools for Use at the Bench’, Cold Spring Harbor Laboratory Press (2002). Each of these general texts is herein incorporated by reference.

Unless defined otherwise, all technical and scientific terms used herein have the same meaning as commonly understood by a person of ordinary skill in the art to which this invention belongs. The following definitions are intended to also include their various grammatical forms, where applicable.

The term “QF”, as used herein, relates to QA-1F, a regulatory gene from the Neurospora crassa qa gene cluster, which is a transcriptional activator that binds to a 16-base pair sequence present in one or more copies upstream of each qa gene.

The term “QS”, as used herein, relates to QA-1S, a regulatory gene from the Neurospora crassa qa gene cluster, which is a repressor of QA-1F (QF) that blocks its transcriptional activity.

The term “transgene”, as used herein, relates to a gene or genetic material that has been transferred by a genetic engineering technique from one organism to another, i.e, the host organism.

The term “transgene expression”, as used herein, relates to the control of the amount and timing of appearance of the functional product of a transgene in a host organism.

Inducible systems are inactive unless there is the presence of a molecule that allows for (induces) gene expression.

Repressible systems are active unless there is the presence of a molecule that suppresses (represses) gene expression.

The term “enhancer-trap screen”, as used herein, denotes a procedure for generating and characterizing transgenes that can be expressed only when integrated close to endogenous DNA regulatory elements that enable the expression of those transgenes.

The term “mosaic”, as used herein, denotes the presence of two populations of cells with different genotypes in one model organism, which has developed from a single fertilized egg. Genetic mosaics in Drosophila melanogaster can be created through mitotic recombination.

MARCM, a mosaic analysis with a repressible cell marker, relates to a genetic mosaic system in Drosophila melanogaster, in which a dominant repressor of a cell marker is placed in trans to a mutant gene of interest. Mitotic recombination events between homologous chromosomes generate homozygous mutant cells, which are exclusively labeled due to loss of the repressor. (Lee & Luo, 1999). MARCM clones can be used to study a mutant phenotype in an otherwise normal animal, or to study the effects of expression of a transgene in the mosaic tissue.

### DETAILED DESCRIPTION

Embodiments of the present invention describe a novel repressible binary expression system ('Q System') based on regulatory genes from the Neurospora qa gene cluster for controlled transgene expression in mammalian and non-mammalian cells as well as in animals.

In one embodiment of the present invention, low basal transgene expression is observed in the absence of the transcriptional activator QF. In another embodiment of the present invention, high transgene expression is observed in the presence of QF. In a further embodiment of the present invention, low basal transgene expression is observed when QF is repressed by its repressor QS. In another embodiment of the present invention, treating Drosophila cells with quinic acid or feeding flies with quinic acid can relieve QS repression.

In further embodiments, methods for lineage tracing, genetic mosaic analysis and for determining gene function are described using the Q system.

### Binary Expression Systems

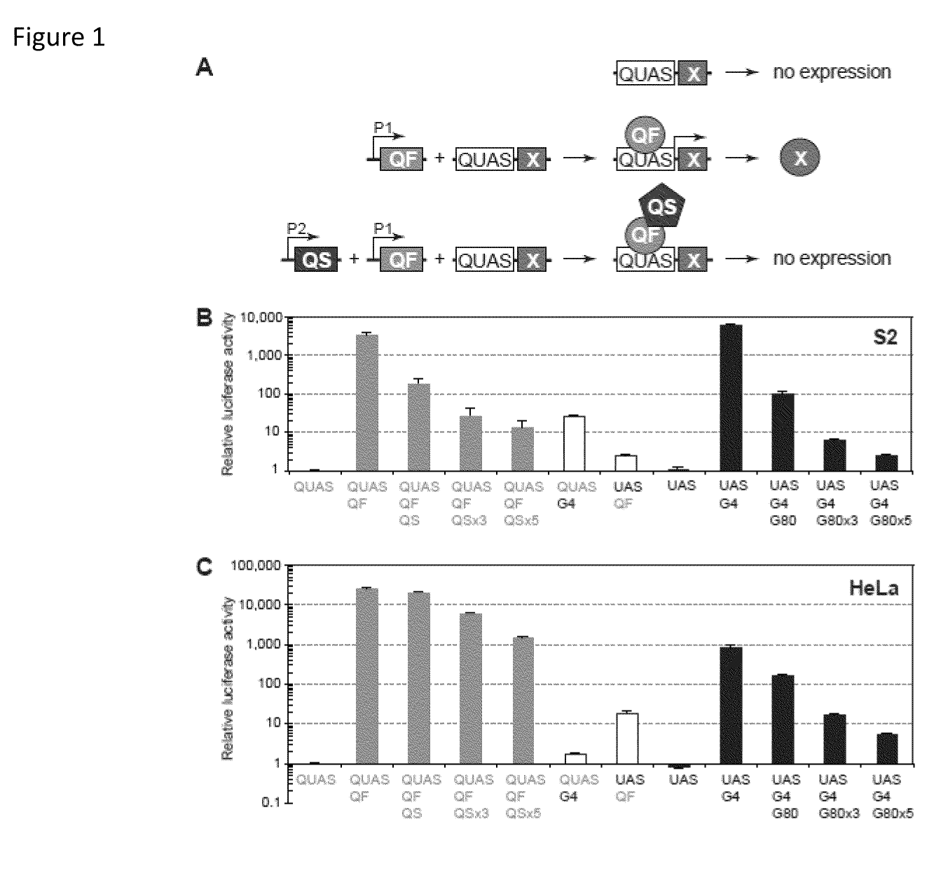

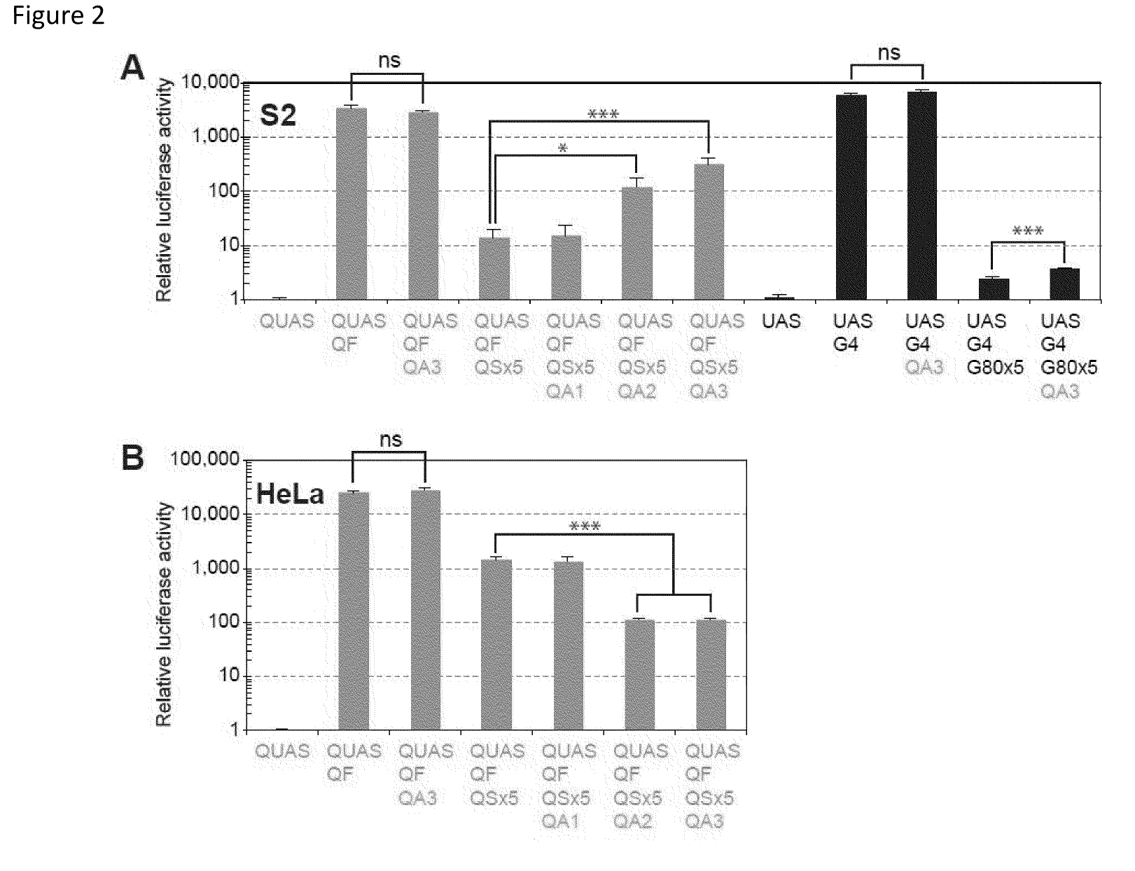

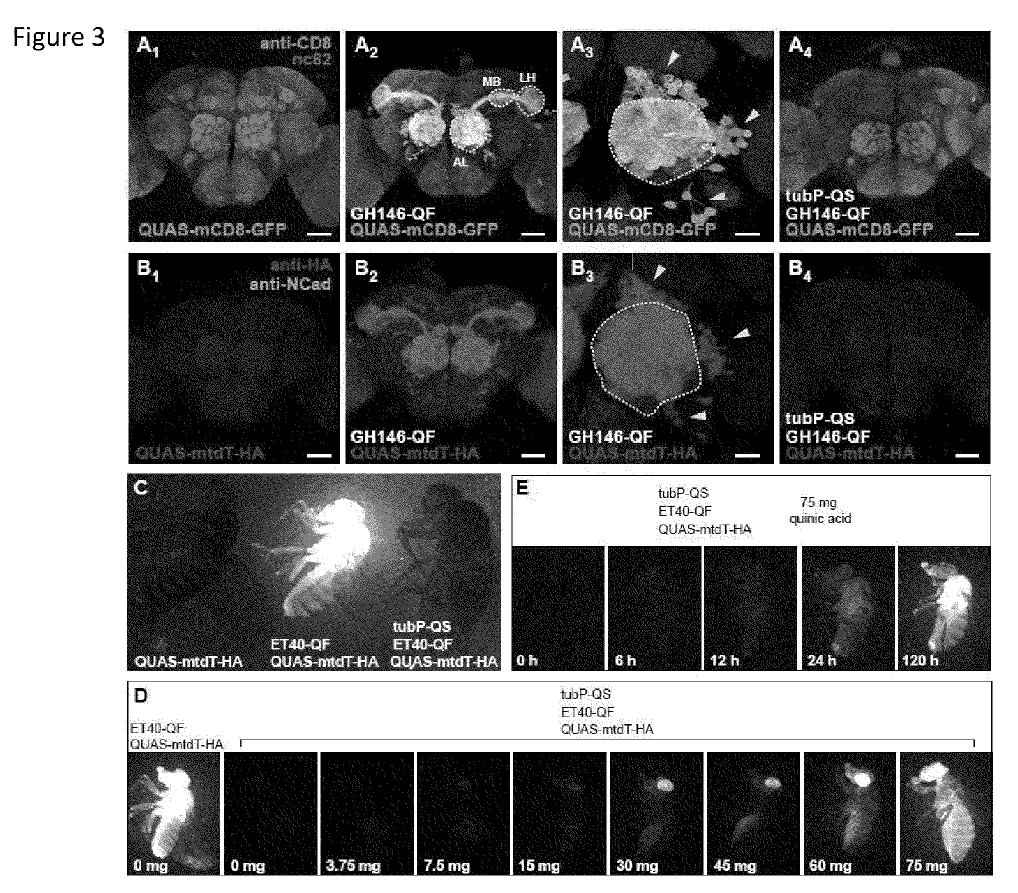

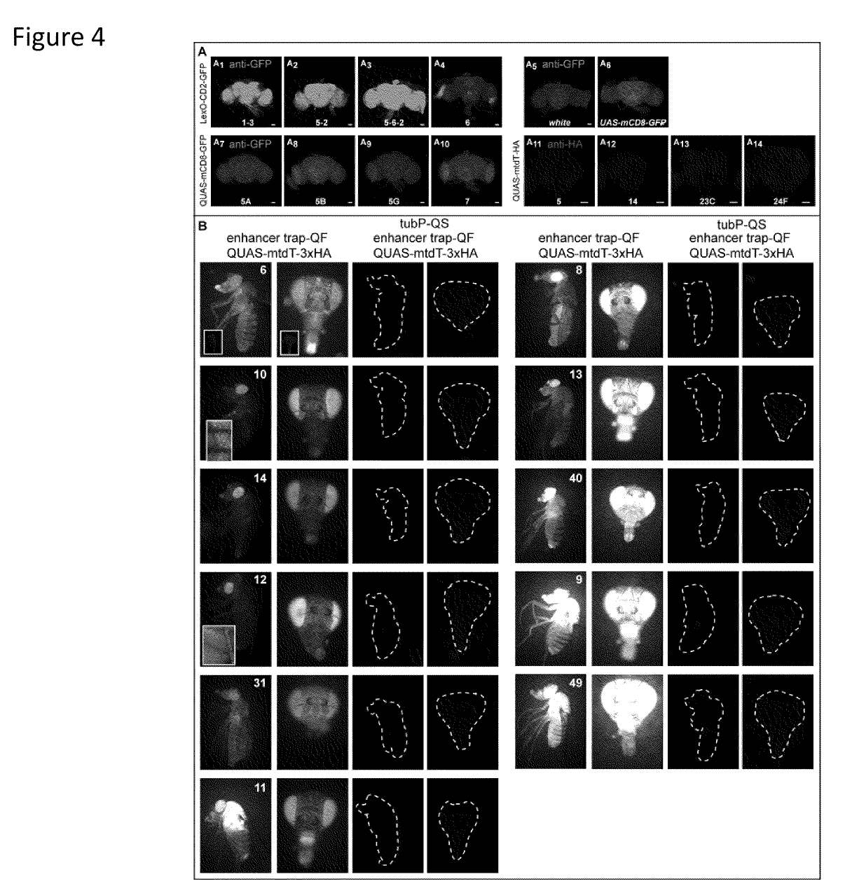

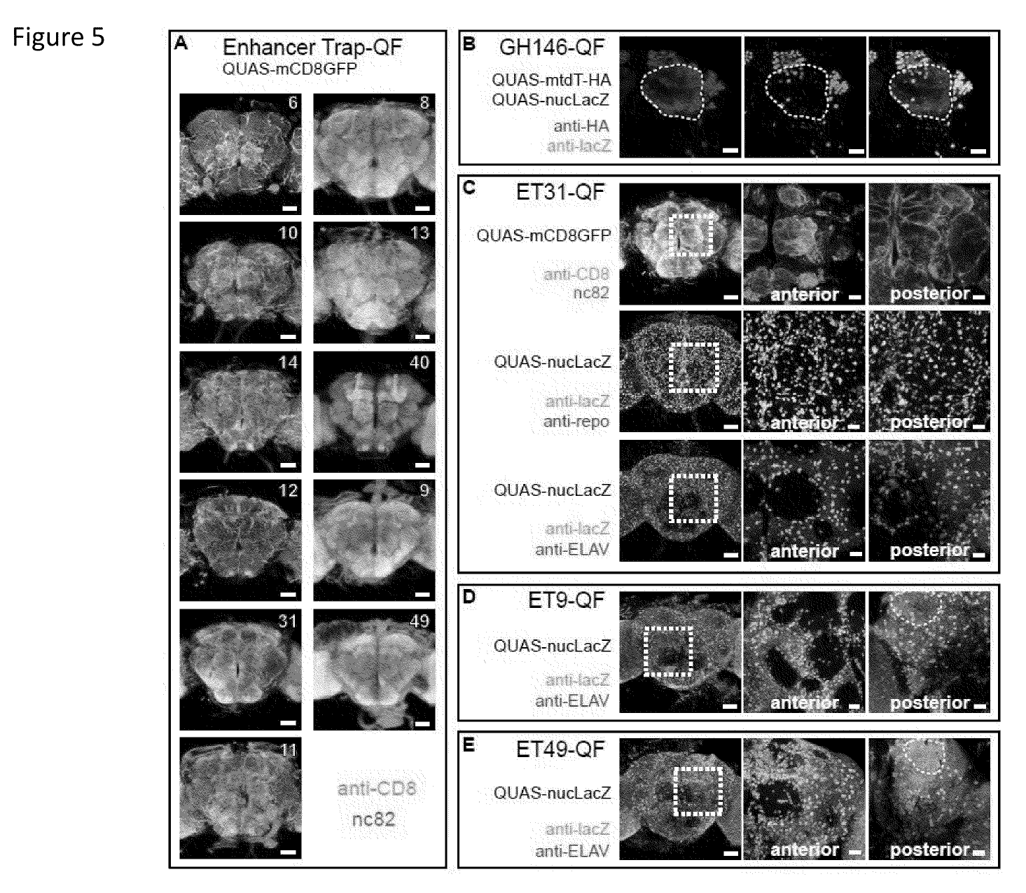

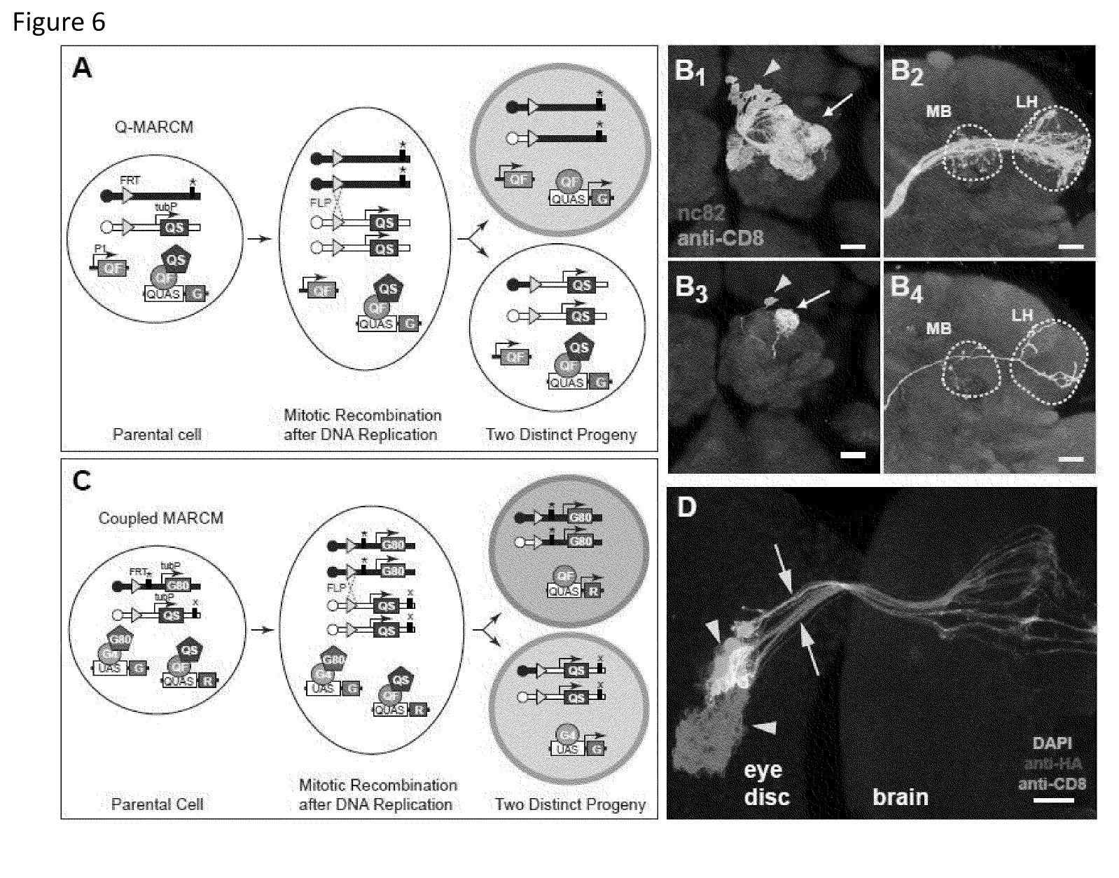

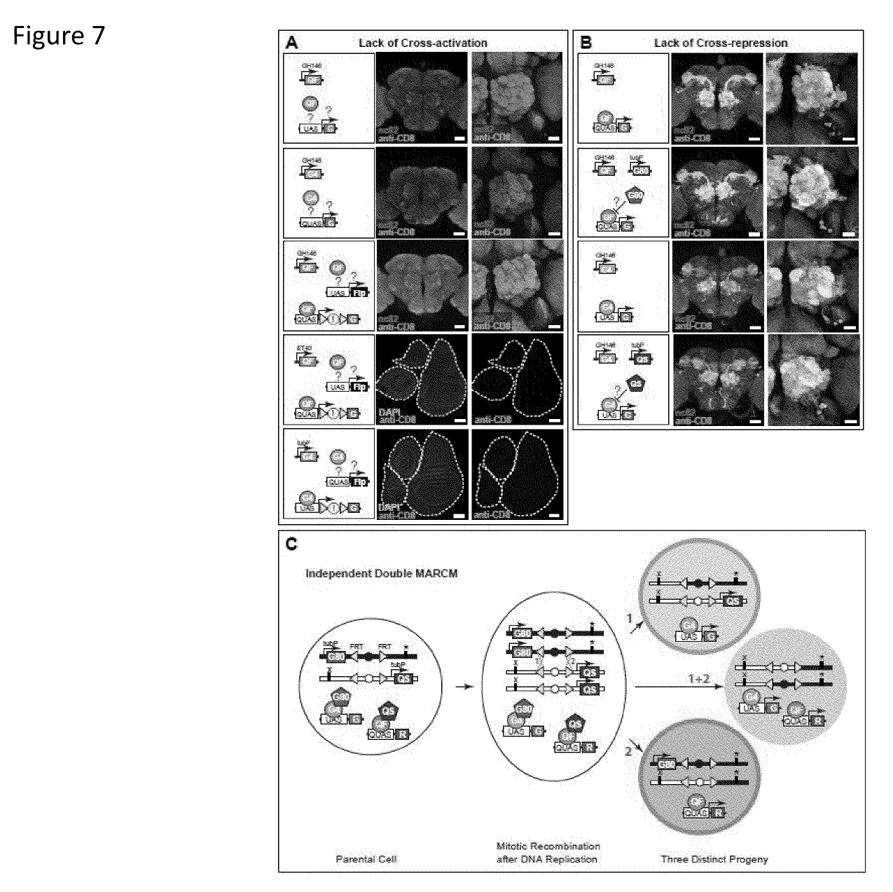

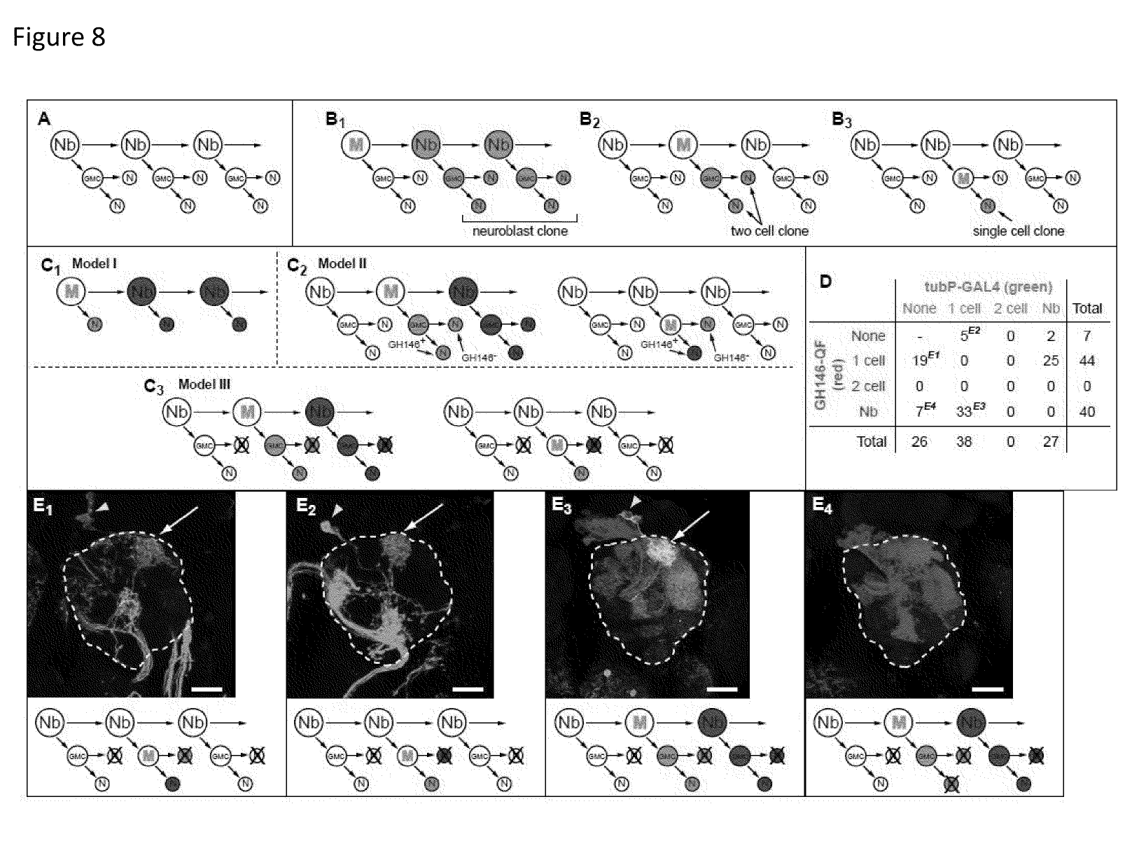

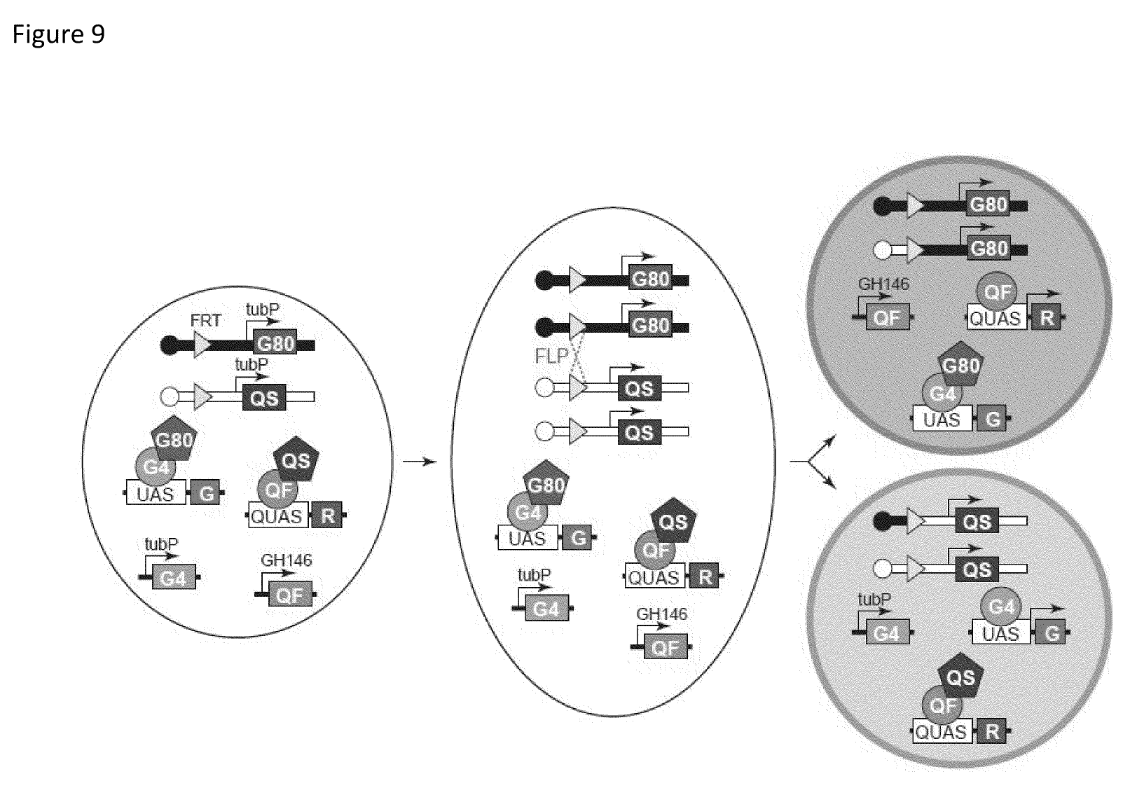

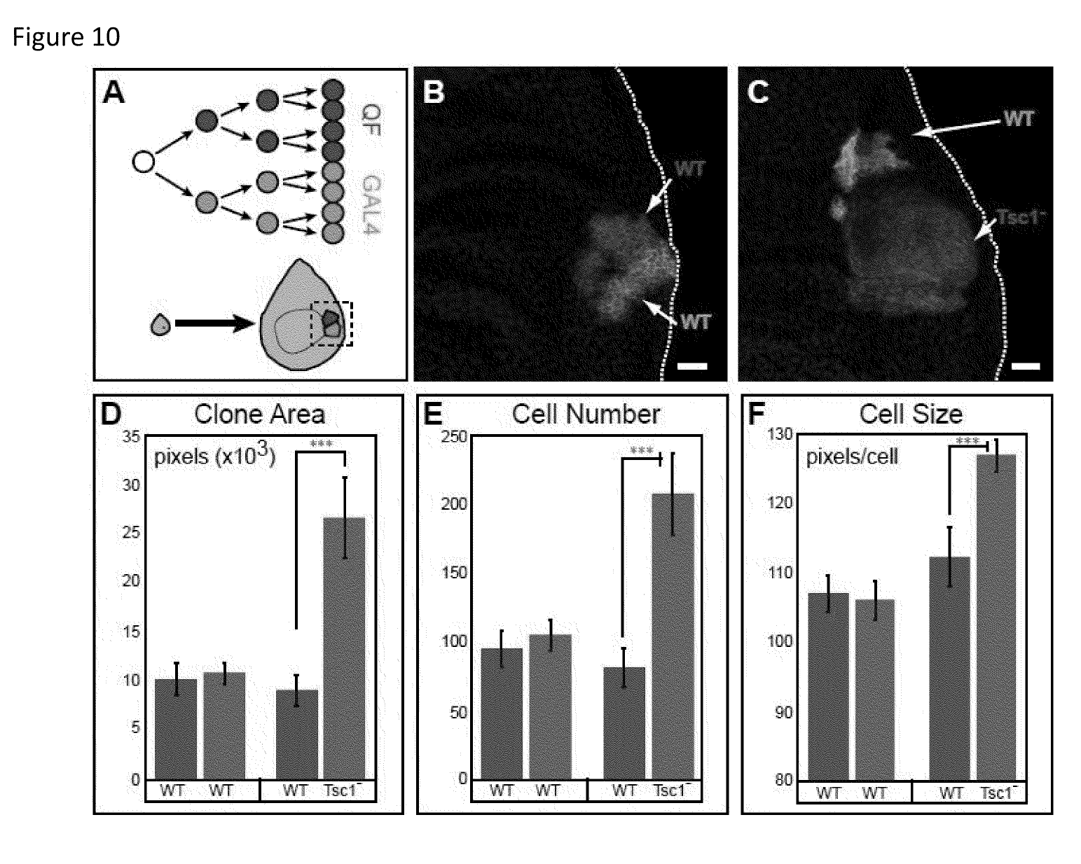

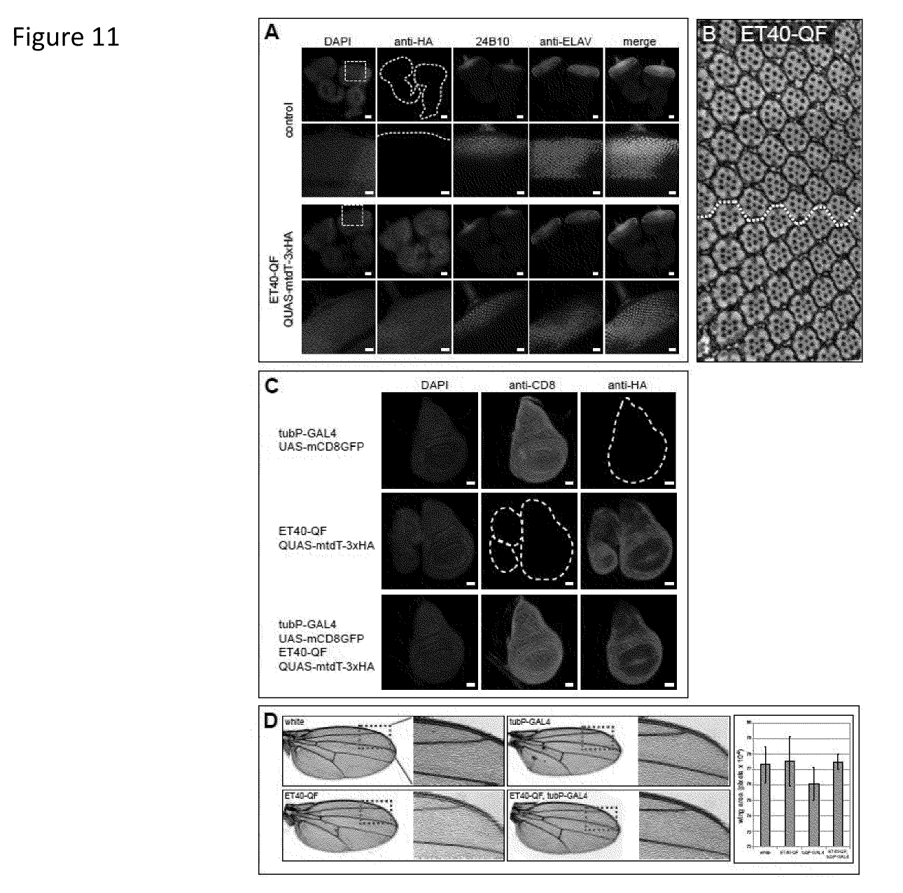

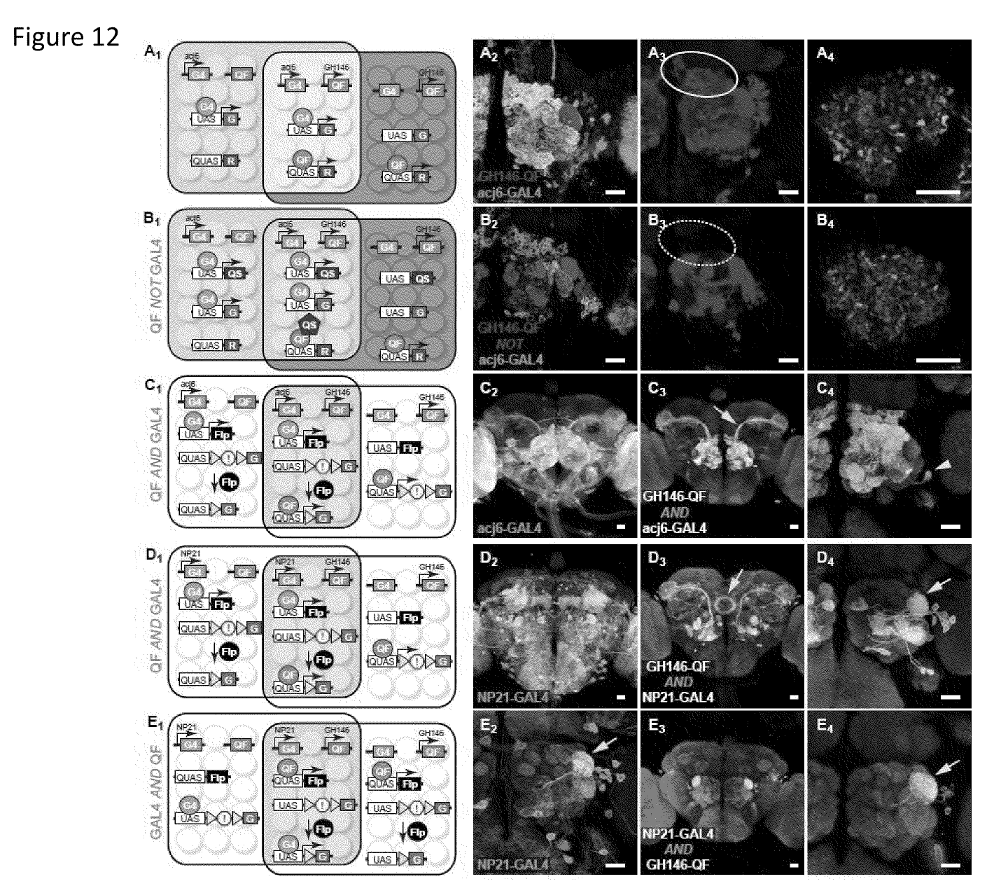

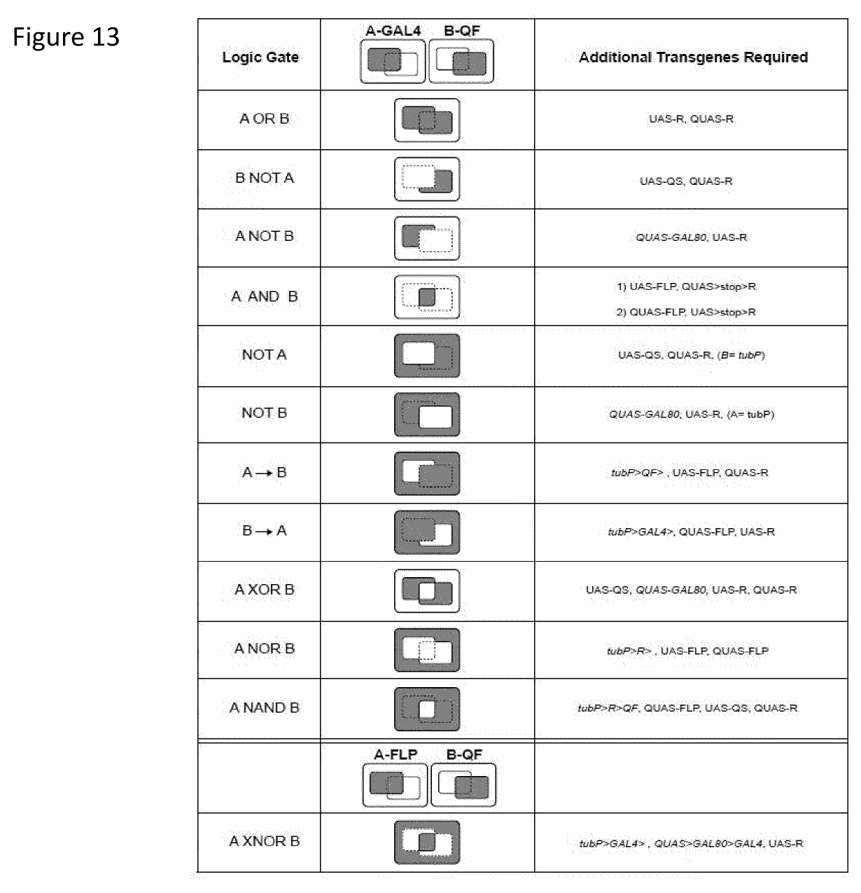

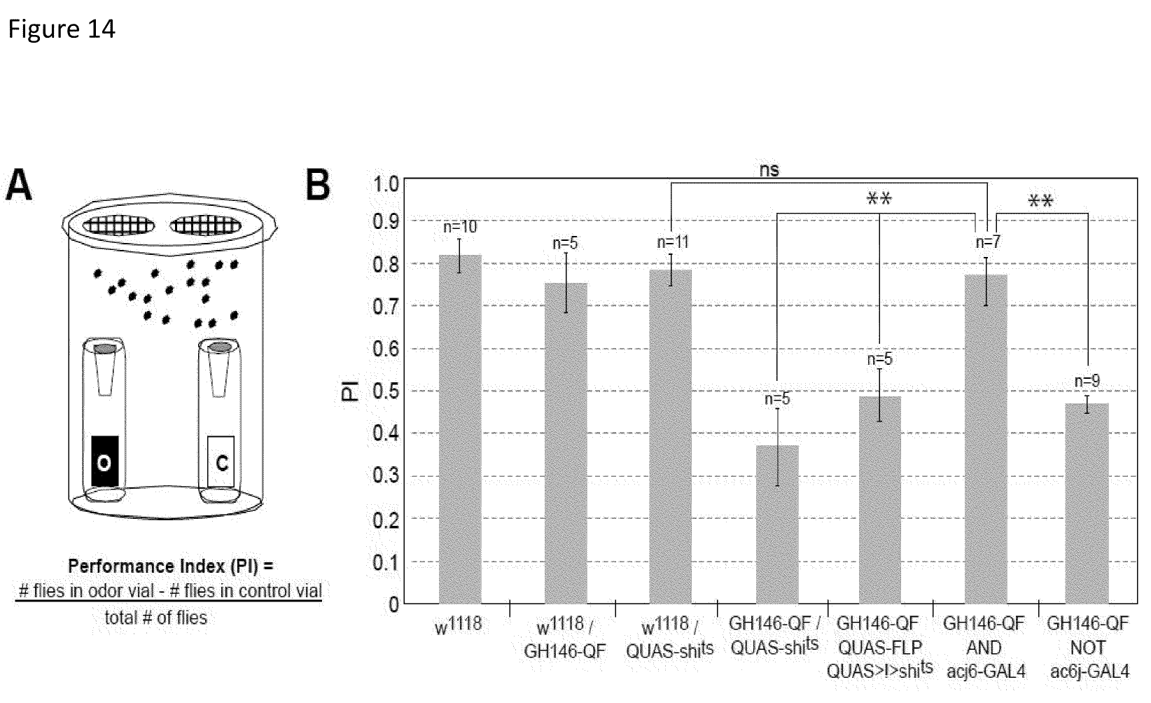

The ability to introduce engineered transgenes with regulated expression into organisms has revolutionized biology. A popular strategy for regulating expression of an effector transgene is to use a binary expression system. In this strategy, one transgene contains a specific promoter driving an exogenous transcription factor, while the other transgene uses the promoter activated only by that transcription factor to drive the effector gene. As a result, the effector gene is controlled exclusively by the chosen transcription factor, and the expression pattern of the effector transgene corresponds to the expression pattern of the exogenous transcription factor (see FIG. 1A).

A number of binary expression systems have been established in genetic model organisms, including tetracycline-regulable tTA/Tetracycline Response Element (TRE) in mice. Compared to effector transgenes driven directly by a promoter, binary systems offer several advantages. First, binary systems usually result in higher levels of effector transgene expression due to transcription factor-mediated amplification. Second, expression of some effectors directly by a promoter may cause lethality and thus prevent the generation of viable transgenic animals. In binary systems, the effector transgene is not expressed until the exogenous transcription factor is introduced into the same animal, usually through a genetic cross. Third, some transcription factors used in binary systems can be additionally regulated by modulators, e.g., small molecule ligands, and thus offer temporal control of transgene expression. Lastly, libraries of transgenes expressing a transcription factor and/or corresponding effectors can be established, such that the transcription factor and effector transgenes can be systematically combined by genetic crosses to enable expression of the same effector transgene in different patterns, or different effector transgenes in the same pattern, thereby enabling genetic screens in vivo.

The impact of the budding yeast-based GAL4/Upstream Activating Sequence (UAS) binary expression system on studies of Drosophila biology cannot be overstated. Thousands of GAL4 lines have been characterized for expression in specific tissues and developmental stages (Brand and Perrimon, 1993; Hayashi et al., 2002; Pfeiffer et al., 2008). Tens of thousands of UAS-effector lines have also been established (Rorth et al., 1998), including a UAS-RNAi library against most predicted genes in the Drosophila genome (Dietzl et al., 2007). In addition to simple binary expression, the finding that the temperature-sensitive yeast repressor of GAL4, GAL80, efficiently represses GAL4-induced transgene expression in Drosophila (Lee and Luo, 1999) offered additional control of the system. For example, in combination with FLP/FRT-mediated mitotic recombination (Golic and Lindquist, 1989; Xu and Rubin, 1993), GAL80/GAL4/UAS can be used to create mosaic animals via MARCM (Lee and Luo, 1999). Using MARCM, mosaic animals can be created that contain a small population of genetically defined cells labeled by a transgenic marker (such as GFP). At the same time, these labeled cells can be homozygous mutant for a gene of interest and/or modified with additional effector transgenes. The MARCM system has been widely used for lineage analysis, for tracing neural circuits, and for high-resolution mosaic analysis of gene function (Luo, 2007).

### Limitations of Existing Binary Expression Systems

The versatile GAL4/UAS system still has limitations. The GAL4 expression patterns from enhancer trap lines or promoter-driven transgenes often include cells other than the cells of interest. It is thus difficult to assign the effect of transgene expression to a specific cell population, especially when phenotypes, such as behavior, are assayed at the whole organism level. Additionally, analysis of gene function and dissection of complex biological systems in multicellular organisms often requires independent genetic manipulations of separate populations of cells. To improve the precision of expression, intersectional expression methods such as the split GAL4 system (Luan et al., 2006) or the combined use of GAL4/UAS and FLP/FRT (Stockinger et al., 2005) have been introduced. To enable independent manipulation of separate populations of cells, additional binary systems such as the lexA/lexAO system have been developed (Lai and Lee, 2006).

Despite extensive efforts to improve the existing binary expression systems, more versatile, precise and controllable systems, as described by embodiments of the present invention, are needed.

### Utility of the Present Invention (“Q-System”)

The Q system utilizes regulatory genes from the Neurospora crassa qa gene cluster. This cluster consists of 5 structural genes and two regulatory genes (QA-1F and QA-1S) used for the catabolism of quinic acid as a carbon source (Giles et al., 1991). QA-1F (shortened as QF hereafter) is a transcriptional activator that binds to a 16-base pair sequence present in one or more copies upstream of each qa gene (Patel et al., 1981; Baum et al., 1987). QA-1S (shortened as QS hereafter) is a repressor of QF that blocks its transactivation activity (Huiet and Giles, 1986) (FIG. 1A). Embodiments of the present invention describe diverse properties of the Q system in fly and mammalian cells, and demonstrate its utility for transgene expression, lineage tracing, cell division analysis and genetic mosaic analysis in Drosophila in vitro as well as in vivo. Moreover, the Q System offers utility for transgene expression, lineage tracing, cell division pattern analysis and genetic mosaic analysis in any organism that is conducive to transgenesis.

Organism Conducive to Transgenesis

In biological research, fruit flies (Drosophila melanogaster) are suitable model organisms to study the effects of genetic changes on development, physiology and behavior. Fruit flies offer the advantages of a short life cycle, low maintenance requirements, and a relatively simple genome compared to higher order organisms. Other suitable model organisms that are conducive to transgenesis include but are not limited to retroviral viruses, bacteria, yeast, flatworms (i.e., nematodes, e.g., C. elegans), fish (zebrafish), mice, rats and monkeys.

Regulation of the Transcription Factor within the Q System by Modulators

Modulators of the transcription factor within the Q System can be added directly to the culture media in in-vitro experiments, while they are administered to experimental animals either by injection or by addition into their food. Modulators can be small molecules, e.g., quinic acid, tetracycline, doxycycline, hormones, hormone analogs (e.g., RU486), proteins, peptides or metal ions.

The Q system offers many applications including: 1) intersectional ‘logic gates’ with the GAL4 system for manipulating transgene expression patterns, 2) GAL4-independent MARCM analysis, 3) coupled MARCM analysis to independently visualize and genetically manipulate siblings from any cell division.

Embodiments of the present invention demonstrate the utility of the Q system in determining cell division patterns of a neuronal lineage and gene function in cell growth and proliferation, and in dissecting neurons responsible for olfactory attraction. The Q system can be expanded to other uses in Drosophila, and to any organism conducive to transgenesis.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible. In the following, experimental procedures and examples will be described to illustrate parts of the invention.

### Experimental Procedures

The following methods and materials were used in the examples that are described further below.

Recombinant DNA Construction

Plasmids were constructed by standard subcloning, synthesis and/or PCR. For PCR amplifications, Phusion Taq polymerase (Finnzymes, Catalog #F530L) was generally used, otherwise Platinum Pfx polymerase (Invitrogen, Catalog #11708-021). When synthetic oligos or PCR were involved in plasmid construction, the sequence of the construct was verified by DNA sequencing.

Three parent plasmids were used for a number of constructs below:

1) pP_{AC5C}-PL (gift of Rui Zhou, Harvard Medical School), which contains Drosophila actin 5c promoter and its polyadenylation sequence. 2) pcDNA3.1-myc-His-A (Invitrogen), which contains human cytomegalovirus (CMV) immediate-early promoter, myc and six-Histidine tags for C-terminal fusion, and bovine growth hormone polyadenylation signal sequence. All cDNAs that were cloned into this plasmid contained a stop codon preceding the Myc and His tags, thereby preventing the fusion of the cDNAs with the tags. 3) pBluescript SK (+) (Stratagene).

pQUAS-GG: This plasmid contains 5 copies of naturally occurring QF binding sites (each 16 bp long, shown in capital letters, with spacer sequences in small letters):

GGGTAATCGCTTATCCtcGGATAAACAATTATCCtcacGGGTAATCGCTT
ATCCgctcGGGTAATCGCTTATCCtcGGGTAATCGCTTATCCtt.

This sequence was assembled from overlapping primers and cloned using XhoI and HindIII into pBluescript containing hsp70 minimal promoter (Brand and Perrimon, 1993) and an optimized GFP split with an intron (GG) (Zong et al., 2005).

pQUAS-luc2: The XhoI/NcoI fragment containing QUAS-Pmin was subcloned from pQUAS-GG into pGL4.23 (Promega) to replace its minimal promoter. pGL4.23 contains synthetic firefly luciferase gene (luc2), which has been codon optimized for high expression in mammalian cells.

pAC-QF: QF cDNA was obtained by PCR using primers PR50 (aatggatcccaacatgccgcctaaacgcaagac) and PR51 (aatgcggccgcctattgctcatacgtgttgatatcg), and the cosmid, pLorist-HO35F3 from the Fungal Genetics Stock Center, as the template. The PCR fragment was cloned into pP_{AC5C}-PL using BamHI and NotI. The QF gene is intronless.

pCMV-QF: Obtained by subcloning QF cDNA using BamHI and Not I from pAC-QF into pcDNA3.1-myc-His-A (Invitrogen).

pCMV-QS: QS cDNA was obtained by PCR using primers PR53 (aatggtacccaacatgaacaccatcccggcac) and PR54 (aatgcggccgctcaagatatttgcgttgcaattc) using a cosmid, pLorist-HO35F3 from the Fungal Genetics Stock Center, as the template. The PCR fragment was initially cloned into pP_{AC5C}-PL using Acc65 I and NotI to obtain the QS gene containing a single intron. The intron was subsequently removed by creating two PCR products that were cloned using 3-way ligation into pcDNA3.1-Mys-His-A (Invitrogen) using Acc65I and NotI and blunt ends at the exon-exon junction. The primers used to create the first exon are: PR53 (see above)+PR190 (agagcctagaggtactctgtgggcgg); and the second exon are: PR58 (tggtcggcgcccaattcc)+PR54 (see above).

pAC-QS: Obtained by subcloning intronless QS from pCMV-QS using Acc65I and NotI into pP_{AC5C}-PL.

pUAS-GG: The BamHIH/BglI fragment from pUAST (Brand and Perrimon, 1993), containing 5 copies of a GAL4 binding site and the Drosophila hsp70 minimal promoter was subcloned into BamHI site of pBluescript containing an optimized GFP split with an intron (GG) (Zong et al., 2005). This strategy generates a hybrid BglII/BamHI site in front of the GFP.

pUAS-luc2: Obtained by subcloning 5 copies of GAL4-binding UAS sequence and the hsp70 minimal promoter from pUAS-GG into pGL4.23 using HindIII and NcoI. The 5xUAS+Pmin fragment from pUAS-GG originates from pUAST (Brand and Perrimon, 1993).

pAC-GAL4 and pCMV-GAL4: GAL4 cDNA was PCR amplified using primers PR509 (CCCCGGATCCCAACatgaagctactgtcttctatcgaaca) and PR510 (CGGTTAACGCGGCCGCttactctttttttgggtttggtg), and a lab vector, pCA-GAL4 as the template. The PCR product was cloned using BamHI and NotI into pP_{AC5C}-PL and pcDNA3.1-myc-His-A, respectively.

pAC-GAL80 and pCMV-GAL80: GAL80 cDNA was PCR amplified using primers PR511 (CCCCGGATCCCAACatggactacaacaagagatcttcgg) and PR512 (CGGTTAACGCGGCCGCttataaactataatgcgagatattgctaacg), and a lab vector, pCA-GAL80 as the template. The PCR product was cloned using BamHI and NotI into pP_{AC5C}-PL and pcDNA3.1-myc-His-A, respectively.

pAC-hRluc: Synthetic Renilla luciferase that was codon optimized for expression in mammalian cells (hRluc) was amplified using primers PR444 (ataaGGTACCaaaATGGCTTCCAAGGTGTACGA) and PR443 (ataaGCGGCCGCTTACTGCTCGTTCTTCAGCA), and pGL4.75 (hRluc/CMV, Promega, catalog #E6931) as the template. The PCR product was cloned into pP_{AC5C}-PL using Acc65I and NotI.

pQUAST: This vector was designed to mimic the multi-cloning site of the pUAST vector (Brand and Perrimon, 1993) thereby allowing easy exchange of inserts between pUAST and pQUAST. pUAST was digested by SphI and EcoRI and blunted with Klenow to remove the 5xUAS and hsp70 minimal promoter (minP). pQUAS-GG was digested with BamHI and EcoRI to excise the 5xQUAS and Pmin and then blunted. The 5xQUAS-Pmin promoter was then ligated into the modified pUAST vector to generate pQUAST. Any gene X can be subcloned from pUAST-geneX into pQUAST using the same restriction sites that were originally utilized for pUAST-geneX construction. If the pUAST-geneX plasmid is not available, genomic DNA from flies containing the UAS-geneX transgene can be used. In this case, pQUAST-geneX can be constructed as follows: 1) PCR amplify the UAS insert from UAS-geneX genomic DNA by using the primer pairs genUASFOR (GCTTCGTCTACGGAGCGACAATTCAATTCAAAC) and genUASREVsv40 (GCAGTAGCCTCATCATCACTAGATGGCATTTCTTC). These primers will amplify the insert for any UAS construct, including the restriction sites used for cloning of that insert into the UAS vector. 2) If the restriction sites used for cloning of the UAS insert are unknown, sequence the PCR fragment using UASFOR-SEQ (TCAAACAAGCAAAGTGAACACG) and SV40REV-SEQ (CCATTCATCAGTTCCATAGGTTGG) primers. 3) Digest the PCR product with appropriate enzymes for cloning into pQUAST.

pQUAST-DSCP: pQUAST was digested with EcoRI, which removed the hsp70 minimal promoter. A PCR fragment containing the DSCP promoter was PCR amplified from pBPGUw (Pfeiffer et al., 2008) with 5′ EcoRI and 3′ MfeI site, and was ligated into pQUAST to generate pQUAS-DSCP. Expression levels between pQUAS-DSCP and pQUAST vectors have not been directly compared (for example, by having the reporter constructs integrated at the same attP location). Nonetheless, pQUAS-DSCP-FLPo and pQUAST-FLPo transgenic flies both showed strong FLP activity in the intersectional studies shown in FIG. 12E.

pmCD8-GFP,y+: This enhancer trap vector is based on the pGalW vector (Gerlitz et al., 2002). The GAL4 insert from pGalW was removed by complete NotI and partial XbaI digestion and replaced with the GAL80 ORF isolated as a NotI/XbaI fragment from pCasper-tubP-GAL80 (Lee and Luo, 1999), to generate the plasmid pG80,w+. To generate pG80,y+, the white gene from pG80,w+ was excised by EcoRV/SacII digestion, the vector was blunted with Klenow, and replaced with the yellow gene as a blunted SalI fragment from the y S/G plasmid (a gift from Pamela Geyer, University of Iowa). To generate pmCD8-GFP,y+, the GAL80 insert was excised by NotI digestion and replaced with mCD8-GFP which was PCR amplified from pUAST-mCD8-GFP to include 5′ and 3′ NotI restriction sites (see also Berdnik et al., 2006).

pQUAST-mCD8-GFP: The NotI fragment containing CD8-GFP-SV40 from the enhancer trap construct pCD8-GFP,y+ was cloned into pQUAST.

ptubP-QS: The QS cDNA was excised from pAC-QS with Acc65I and NotI, blunted, and ligated into a blunted pCasper-tubulin-GAL80, from which the GAL80 insert was removed by digestion with NotI/XbaI.

pBac-GH146-QF: pBAC-3xPDsRed-GH146-MCS with FseI-AscI-AvrII multi-cloning site has been previously described (Hong et al., 2009). QF cDNA with 5′ FseI and 3′ Avr restriction sites was amplified from pAC-QF by PCR, and cloned into the FseI/AvrII restriction sites of pBAC-GH146 to yield pBac-GH146-QF.

pQUAST-mtdT-3xHA, pUASTattB-mtdT-3xHA: The N-terminal membrane tag on tdTomato contains 8 amino acids that direct myristoylation and palmitoylation (Muzumdar et al., 2007). 3 copies of the HA tag were PCR amplified from pTHW (Drosophila Genomics Resource Center) and included a 5′ BsrGI restriction site and a 3′ EcoRI restriction site preceded by the TAA stop codon (5′ oligo:TTATGTACAAGTACCCATACGATGTTCCTGACTATGC; 3′ oligo: TAAGAATTCTTAAGCGTAATCTGGAACGTCATATGGATAGG). pSN20-mtdT (Muzumdar et al., 2007) was digested with BsrGI and EcoRI and the HA PCR fragment was ligated into this vector to generate pSN20-mtdT-3xHA. This vector was then digested with XhoI and partially digested with BamHI and cloned into pQUAST and pUASTattB (Bischof et al., 2007) to generate pQUAST-mtdT-3xHA and pUASTattB-mtdT-3xHA respectively. The mtdT-3xHA reporter in vivo is as good as mCD8-GFP in labeling dendritic and axonal processes, or in labeling imaginal disc tissues. However, it does not label neuronal cell bodies as well as mCD8-GFP as most of the mtdT-3xHA signal is localized to the plasma membrane surface whereas mCD8-GFP, which also localizes to intracellular membranes, allows for the cell soma to be better visualized.

pQUAST-nucLacZ: The nuclear LacZ insert was PCR amplified from UAS-nucLacZ genomic flies (Bloomington Stock Center) using oligos genUASFOR and genUASsv40REV. To determine which cloning sites were used in the cloning of UAS-nucLacZ, the PCR fragment was sequenced using UASFOR-SEQ and SV40REV-SEQ. The KpnI/XbaI digested nucLacZ PCR fragment was then ligated into pQUAST.

pQUAST-FLPo: Mammalian codon-optimized FLP recombinase (FLPo; Addgene plasmid 13792) (Raymond and Soriano, 2007) was PCR amplified to include BglII/NotI restriction sites, and cloned into pQUAST to generate pQUAST-FLPo.

pQUAS-DSCP-FLPo: Mammalian codon-optimized FLP recombinase (FLPo; Addgene plasmid 13792) (Raymond and Soriano, 2007) was PCR amplified to include EcoRI/XbaI restriction sites, and cloned into pQUAS-DSCP to generate pQUAS-DSCP-FLPo.

pCa4B2G-QUAS-DSCP-FLPo: The QUAS-DSCP-FLPo-SV40 region was excised from pQUAS-DSCP-FLPo by BamHI digestion, and subcloned into pCa4B2G at the BamHI site that is flanked by gypsy insulators (Markstein et al., 2008). This construct was used for PhiC31-mediated integration into the attP2 locus (Groth et al., 2004).

pUAST>stop>mCD8-GFP: The widely used FLP-Out reporter, UAST>CD2,y+>mCD8-GFP (Wong et al., 2002) contains non-optimal FRT sites (‘>’ represents FRT), which were chosen in order to increase the percentage of small FLP-out clones during FLP-out labeling experiments (G. Struhl, personal communication). As such, in control FLP experiments, there was some variability in the extent of the excision of the ‘CD2,y+’ cassette in all expected target neurons, presumably due to incomplete excision of the FLP-out cassette. To reduce this variability, optimal FRT sites were used to flank two transcription stops. Direct comparison between UAS>stop>mCD8-GFP and UAS>CD2,y+>mCD8-GFP transgenic flies—in test crosses with GH146-FLP (Hong et al., 2009) and elav-GAL4—verified that the new FLP-out reporter is more effective in removing the cassette between the FRT sites, resulting in decreased variability in mCD8-GFP reporter expression (data not shown). Construction of UAST>stop>mCD8-GFP has been previously described (Hong et al., 2009).

pQUAST>stop>mCD8-GFP: The FRT-Stop-FRT cassette from pUAS>Stop>mCD8-GFP was excised by BglII/NotI digestion. The mCD8-GFP cassette was excised from pQUAST-mCD8-GFP by NotI digestion. The two inserts were ligated into the BglII/NotI sites of the pQUAST vector.

pQUAST-shibire^{ts1}: Shibire^{ts1} was PCR amplified from genomic DNA of UAS-shibire^{ts1} transgenic flies (Kitamoto, 2001) using genUASFOR and genUASREVsv40 oligos, and ligated into the NotI/KpnI sites of pQUAST. To test for functional transgenic QUAS-shibire^{ts1} insertions, we crossed flies containing a particular insertion with flies carrying ET49-QF, which broadly expresses QF in many neurons, and then placed the progeny in a 37° C. water bath. Flies with a functional QUAS-shibire^{ts1} became paralyzed within 20 seconds, followed by full recovery within 5 minutes.

pQUAST>stop>shibire^{ts1}: The NotI fragment containing the mCD8-GFP cassette was excised from pQUAS>stop>mCD8-GFP, the vector was blunted, and ligated to a blunted NotI/KpnI shibire^{ts1} isolated from pQUAST-shibire^{ts1}. Functional transgenic QUAS>stop>shibire^{ts1} insertions were tested by crossing them to hsFLP122 and ET49-QF. The progeny containing all three transgenes were heat shocked at least once during development to excise the >stop>cassette, and then tested as described for QUAS-shibire^{ts1}.

pQF-M2ET (swappable QF enhancer trap): An enhancer trap was constructed that allows recombination mediated cassette exchange (Oberstein et al., 2005). A loxM2 site was inserted immediately following the 5P promoter, and a LoxP site was placed after the selectable white marker to generate the vector pDonor-M2, which contains elements in the following order: 5P-Ppromoter-LoxM2-MCS-SV40 terminator-white-LoxP-3P. QF was PCR amplified from pAC-QF adding 5′ EcoRI and 3′ AatII restriction sites, and inserted into the EcoRI/AatII cloning sites of pDonor-M2 to yield pQF-M2ET. Of the 14 original insertions of pQF-M2ET from the initial injection, 5 showed only tracheal expression, 8 showed tracheal expression and expression in additional tissues, and only 1 (ET40) showed very minor tracheal expression and high expression in additional tissues (e.g. all imaginal discs, and some adult brain structures). The reason for the tracheal expression is currently unknown. We are generating additional enhancer trap vectors to circumvent the tracheal expression. The ET40 enhancer trap is inserted into the 5′ upstream region of the posterior sex combs (psc) gene located at 49E6. The name, cytological location, and associated gene of the pQF-M2ET insertions in FIG. S2B are: #6, 78A2, skuld; #8, 47A7, lola; #31, 52E11, Ext2.

pQF-ET (QF enhancer trap): The QF-SV40polyA fragment was excised from pQF-M2ET,w+ by digestion with EcoRI and BamHI, blunted by Klenow and inserted into blunted pGalW (Brand and Perrimon, 1993) from which the GAL4-hsp70polyA fragment had been excised by NotI digestion. The name, cytological location, and associated gene of the pQF-ET insertions in FIG. S2B are: #11, 85C3, CG11033; #9, 49E7, Su(z)2; #12, 34A10, snRNA:U2; #13, 44B5, kermit; #14, 39A5, intergenic; #10, 34A10, CG9426; #49, 70C15, Hsc70b.

pUASTattB-QS: The QS cDNA was excised from pAC-QS with Acc651 and NotI, blunted, and ligated into a blunted pUASTattB (Bischof et al., 2007) which had been digested with BglII and KpnI. Transgenic flies with this construct integrated into the attP2 site were used in the experiments shown in FIG. 12B.

Constructs for the Generation of Additional Q System Reagents

pattB-QF-sv40: The QF-sv40 cassette fom pQF-M2ET is PCR amplified to include EcoRI/NotI restriction sites and inserted into the pattB vector (Bischof et al., 2007).

pattB-QF-hsp70: The QF-hsp70 cassette from pBac-GH146-QF is PCR amplified to include EcoRI/NotI restriction sites and inserted into the pattB vector (Bischof et al., 2007).

pattB-DSCP-QF-sv40: The DSCP from pBPGUw (Pfeiffer et al., 2008) is PCR amplified to include EcoRI/MfeI restriction sites and ligated into the EcoRI site of pattB-QF-sv40.

The previous three vectors contain convenient multi-cloning sites preceding the QF ORF for cloning of promoter/enhancer regions to drive QF expression. These QF constructs can be integrated using PhiC31 mediated integration (Bischof et al, 2007). The hsp70 terminator results in reduced QF expression (in comparison to QF-sv40) and allows for transformation of constructs that proved toxic with QF-sv40 (Potter and Luo, unpublished observations). The DSCP variant can be used if the cloned enhancer region does not contain a promoter (Pfeiffer et al., 2008). These constructs can also be used for convenient isolation of the QF ORF by restriction digestion.

pBS-SK-QS: The QS ORF is PCR amplified from pAC-QS to include EcoRI/XbaI restriction sites, and cloned into pBluescript-SK (Strategene). This construct contains convenient restriction sites before and after QS and can be used for cloning of QS promoter/enhancer constructs or for isolation of the QS ORF.

Expression Studies in Cultured Cells

S2 Cell Transfection: S2 cells (gift of M. Simon, Stanford University) were maintained in Shields and Sang M3 insect medium (Sigma, Catalog #S8398), prepared according to the manufacturer's instructions and supplemented with 10% heat inactivated fetal bovine serum (FBS) and antibiotics (from 100× penicillin/streptomycin stock, Invitrogen, Catalog #15140). FBS was heat inactivated at 56° C. for 30 minutes. The cells were grown in an air incubator at 25° C. For transfection, 0.4 ml of complete M3 medium containing 5*10⁵ Drosophila S2 cells were plated into individual wells of 24-well plates several hours before the transfection. The DNA for transfection was mini-prepped (QIAprep Miniprep kit, Qiagen, Catalog #27106), DNA concentrations were determined using the Nanoprop 1000 spectrophotometer (Thermo Scientific), diluted in 10 mM TRIS-HCl pH 7.5, 0.1 mM EDTA to 25 ng/μl, and filtered through a 13-mm 0.2 μm filter (Nalgene, Catalog #180-1320). For individual transfections, we used 0.2 μg of total DNA (total of 8 μl from 25 ng/μl stocks) including one or more of the following: 25 ng of reporter, 12.5 ng of a transcription factor plasmid and the amount or repressor plasmid that was adjusted according to the molarity of the corresponding transcription factor plasmid to get either equimolar, 3-fold or 5-fold higher molar concentration, as indicated.

Each sample also contained 2.5 ng of pP_{AC5C}-hRluc, which was used for normalization of the firefly luciferase signal for each sample (see below) and pBluescript to supplement the total DNA amount to 0.2 μg. The cells were transfected using Effectene (Qiagen, Catalog #301425) according to the manufacturer's protocol (for each sample we used: 59 μl EC buffer; 1.6 μl enhancer solution; 4 μl Effectene and 350 μl M3 medium). 12 h after the addition of transfection mixes to cells, quinic acid was added where indicated from 50× stocks. The starting quinic acid stock solution (250 mg/ml) was prepared from D-(−)-quinic acid (Sigma-Aldrich, 98%, Catalog #138622) in sterile Milli-Q water and neutralized with NaOH to pH˜7. All other 50× stocks were made from this stock by dilution in water. Cells were lysed 24 h after the addition of quinic acid (36 h since the start of transfection), by spinning the 24-well plates for 5′ in a table top centrifuge at ˜1000 g, removing the supernatant by aspiration and incubation with of 200 μl passive lysis buffer (from the Dual Luciferase Reporter Assay System by Promega, Catalog #E1910) with shaking at room temperature for ˜30 minutes. The lysates were transferred into 1.5 ml tubes and frozen at −80° C. The lysates were analyzed using the Dual Luciferase Reporter Assay System, according to the manufacturer's instructions, and the single tube Turner Biosystems luminometer, model 20/20n. The luminescence light signal for both firefly and Renilla luciferases was collected for 10 s. All samples that were transfected with reporters had at least 100-fold higher signal than the background (lysates from pBluescript-only transfected cells). For each sample “X”, the relative luciferase activity (RLA) was calculated according to the following formula:

$${RLA}_{x} = ( {F}_{x} / {R}_{x} ) / {( \overline{F / R} )}_{QUAS} , \\ where , \\ {( \overline{F / R} )}_{QUAS} = ( {\sum }_{i = 1}^{n} {F}_{QUAS}^{i} / {R}_{QUAS}^{i} ) / n$$

where n=number of QUAS-only samples; F=Firefly luciferase luminescence signal; R=Renilla luciferase luminescence signal. Each condition was executed at least in triplicate, the average and SEM were determined for each condition, and statistical significance was evaluated using Student's t-test. Plasmids used for transfection in S2 cells were: pUAS-luc2, pQUAS-luc2, pAC-QF, pAC-QS, pAC-GAL4, pAC-GAL80, pAC-hRluc, pBluescript.

Full suppression was not observed with equimolar ratios of QF and QS (similar lack of full suppression was observed with GAL4/GAL80). This result could be a consequence of transient transfections, where individual cells do not necessarily uptake the same amounts of activator and repressor plasmids. Additionally, somewhat higher QS/QF ratios compared to GAL80/GAL4 ratios are required to reach similar degree of repression. The difference between the two systems probably lies in the specific nature of the individual genes or the corresponding proteins (activity, codon choice, mRNA or protein stability, etc.).

HeLa Cell Transfection: HeLa cells (gift of K. Wehner, Stanford University) were maintained in Dulbecco's Modified Eagle Medium (DMEM, Invitrogen, Catalog #10566), supplemented with 10% FBS and antibiotics (from 100× penicillin/streptomycin stock, Invitrogen, Catalog #15140) in a 37° C. air incubator with 5% CO₂. At least 5 hours before transfection, cells were detached and dissociated from the plate using Triple-express (Invitrogen, Catalog #12605) and plated into 24-well plates at 7*10⁴ cells in 0.5 ml medium with FBS, but without antibiotics. Cells were transiently transfected using Lipofectamine 2000 (Invitrogen, Catalog #11668-019), according to the manufacturer's instructions. Plasmid DNA for transfection was prepared as described in the S2 cell transfection protocol above. For each sample we used 0.25 μg DNA (total of 10 μl from 25 ng/μl stocks) including one or more of the following: 50 ng of reporter, 25 ng of a transcription factor plasmid and the amount or repressor plasmid that was adjusted according to the molarity of the corresponding transcription factor plasmid to get either equimolar, 3-fold or 5-fold higher molar concentration, as indicated.

Each sample also contained 5 ng of pGL4.75 (hRluc/CMV from Promega), which was used for normalization of the firefly luciferase signal for each sample (see below), and pBluescript to supplement the total DNA amount to 0.25 μg. The DNA was mixed in 50 μl of Opti-MEM (Invitrogen, Catalog #51985) and combined with another 50 μl of Opti-MEM containing 1 μl of Lipofectamine. The medium containing the transfection mix was replaced with DMEM containing FBS and antibiotics 22 h after transfection. Where needed, the medium contained quinic acid supplemented from 50× stocks that were made as described in the S2 cell transfection protocol above. 24 h after the medium was changed, the cells were lysed in 24-well plates and processed as described above in the S2 cell transfection protocol. All samples that were transfected with reporters had at least 100-fold higher signal than the background (lysates from pBluescript-only transfected cells). We have noticed that significantly poorer repression of QF by QS is obtained with shorter transfection times in mammalian cells. Plasmids used for transfection in HeLa cells were: pUAS-luc2, pQUAS-luc2, pCMV-QF, pCMV-QS, pCMV-GAL4, pCMV-GAL80, pGL4.75 (Promega), pBluescript. Higher QS:QF or GAL80:GAL4 molar ratios are required for effective suppression in HeLa cells compared with Drosophila S2 cells. This phenomenon could be due to codon choice and/or differential activity or stability of activators and repressors at different temperatures—25° C. for S2 and 37° C. for HeLa.

Drosophila Genetics and Manipulations

Transgenes were generated by standard P-element (Spradling and Rubin, 1982) or PhiC31 integrase-mediated transformation (Groth et al., 2004), as noted in Table 1. All fly transgenes were mapped using splinkerette PCR. When necessary, transgenes were recombined onto the same chromosome using standard meiotic recombination techniques. To check for TscI^{Q600X} (Potter et al., 2001) recombinants, a genomic region containing the Tsc1 point mutation was PCR amplified from genomic DNA using oligos gTsc1-FOR#1 (GCTGCAGTTTGTGGCGAGTG) and gTsc1-REV#1 (AACCGATCCCGCTCCATTTC) and cut with the restriction enzyme SnaBI. The SnaBI site was created by the C->T mutation in the Tsc1^{Q600X} allele. Wildtype Tsc1 gives an uncut band of 719 bp and the Tsc1^{Q600X} mutant gives bands of sizes 322 bp and 397 bp.

TABLE 1
Q System Resources - Transgenics and Recombinants
Generated in Embodiments of the Present Invention

| Name | Chromosome ^{a} |
|---|---|
| QUAS reporters | |
| QUAS-mCD8-GFP | X, 2^{nd}, 3^{rd} |
| QUAS-mtdTomato-3xHA | X, 2^{nd}, 3^{rd} |
| QUAS-nucLacZ | X, 2^{nd}, 3^{rd} |
| QUAS-FLPo | X, 2^{nd}, 3^{rd}, |
| | 3^{rd} (attP2 ^{b}) |
| QUAS>stop>mCD8-GFP | 2^{nd}, 3^{rd} |
| QUAS-shibire^{ts1} | X, 2^{nd}, 3^{rd} |
| QUAS>stop>shibire^{ts1} | 3^{rd} |
| QF lines: | |
| GH146-QF | 2^{nd}, 3^{rd} |
| ET9-QF | 2^{nd} |
| ET31-QF | 2^{nd} |
| ET40-QF | 2^{nd} |
| ET49-QF | 3^{rd} |
| QS lines: | |
| tubP-QS | X, 2L, 2R, |
| | 3L, 3R ^{c} |
| UAS reporters: | |
| UAS-QS | 3^{rd} (attP2, |
| | ZH-attP-86Fa ^{d}) |
| UAS>stop>mCD8-GFP | 2^{nd}, 3^{rd} |
| UAS-mtdTomato-3xHA | 3^{rd} (ZH- |
| | attP-86Fa) |
| Recombinant Transgenic Animals: | |
| GH146-QF recombinants: | |
| GH146-QF#53, QUAS-mCD8-GFP | 3^{rd} |
| GH146-QF#53, QUAS-mtdT-3xHA | 3^{rd} |
| GH146-QF#11, UAS>stop>mCD8-GFP | 2^{nd} |
| GH146-QF#11; UAS>stop>mCD8-GFP | 2^{nd}, 3^{rd} |
| GH146-QF#11, UAS-FLP | 2^{nd} |
| y, w; Pin/CyO; UAS-FLP, GH146-QF#53 | 3^{rd} |
| GH146-QF#11; QUAS-FLPo^{attp2} | 2^{nd}, 3^{rd} |
| GH146-QF#53 FRT^{82B} | 3^{rd} |
| GH146-QF#53, FRT^{82B}, tubP-QS#9B | 3^{rd} |
| GH146-QF#53, FRT^{82B}, tubP-QS#21 | 3^{rd} |
| ET40-QF recombinants: | |
| ET40-QF, QUAS-mCD8-GFP | 2^{nd} |
| ET40-QF, QUAS-mtdT-3xHA | 2^{nd} |
| Stocks for MARCM analysis: | |
| tubP-QS#4, 40A^{FRT} | 2^{nd} |
| FRT^{82B}, tubP-QS#9B | 3^{rd} |
| FRT^{82B}, tubP-QS#21 | 3^{rd} |
| tubP-GAL4, FRT^{82B}/TM6B | 3^{rd} |
| y, w; Pin/CyO; tubP-GAL4, FRT^{82B}/TM6B | 3^{rd} |
| y, w; Pin/CyO; tubP-GAL4, FRT^{82B}, tubP-GAL80/ | 3^{rd} |
| TM6B | |
| y, w; Pin/CyO; tubP-GAL4, FRT^{82B}, tubP-GAL80, | 3^{rd} |
| Tsc1^{Q600X}/TM6B | |
| y, w; ET40-QF, QUAS-mtdT-3xHA; FRT^{82B}, tubP- | 2^{nd}, 3^{rd} |
| QS#9 | |
| y, w; ET40-QF, QUAS-mtdT-3xHA; FRT^{82B}, tubP- | 2^{nd}, 3^{rd} |
| QS#21 | |
| hsFLP, QUAS-mtdT-3xHA, UAS-mCD8-GFP | X |
| UAS-mCD8-GFP, QUAS-mtdT-3xHA; Pin/CyO | X |
| QUAS-mCD8-GFP#5B, tubP-GAL4/TM6B | 3^{rd} |
| acj6-GAL4, UAS-mCD8-GFP/FM7c; Pin/CyO | X |
| y, w; FRT^{82B}, Tsc1^{Q600X}/TM6B | 3^{rd} |

^{a} See Schematic for cytological locations of insertion sites for a subset of these transgenes.
^{b} See (Groth et al., 2004; Markstein et al., 2008).
^{c} These transgenes have been recombined with common FRTs on respective
chromosomes.
^{d} See (Bischof et al., 2007).

Toxicity of QF: We were unsuccessful in generating tubP-QF transgenic animals by either site-directed integration using PhiC31 integrase or P-element mediated transformation. These observations suggest that QF is toxic to flies when highly expressed in a ubiquitous manner or in a particular developmental stage or tissue. We note that in our previous effort to generate tubP-GAL4, we encountered similar difficulty in obtaining transgenes from microinjection or transposition. In parallel experiments, we obtained a single tubP-GAL4 transgene but more than a dozen tubP-GAL80 transgene insertions (Lee and Luo, 1999). Furthermore, repeated experiments of remobilizing the tubP-GAL4 transgene resulted in only one additional line on the same chromosome. Both tubP-GAL4 transgenes are homozygous lethal, but become homozygous viable in the presence of a tubP-GAL80 transgene, suggesting that high-level ubiquitous expression of GAL4 is toxic. We suspect that high-level ubiquitous expression of strong transcription factors such as GAL4 and QF is harmful to flies—probably due to squelching of the transcriptional machinery. It is also possible that high-level expression of QF in some tissues and developmental stages is more harmful than equivalent expression of GAL4.

To address the described toxicity issue, for instance, we have generated a mutant QF protein (QFm) that exhibits ˜5 fold less activity in S2 transfection assays compared to wild-type QF (BT and LL, unpublished results). We were able to obtain tubP-QFm transgenic animals, as well as >100 QFm enhancer trap lines, but reporter expression levels were not sufficiently strong in many tissues (CJP and LL, unpublished results). We are in the process of isolating a QF variant that drives report expression sufficiently well in all tissues. We are also conducting experiments to address the possibility that a particular developmental stage or a particular tissue is especially sensitive to QF expression. Nonetheless, we have isolated many QF enhancer trap lines that express strongly in many tissues without adverse effects, including imaginal discs (ET40-QF), glia (ET31-QF), trachea (ET14-QF) and neurons (GH146-QF and ET49-QF), suggesting that high-level expression of QF is not toxic to many cell types. We further note that the toxicity associated with high-level ubiquitous expression of GAL4 does not prevent the widespread use of the GAL4/UAS system.

Coupled MARCM analysis of projection neuron lineage: Vials containing approximately 15 males and 25 female fruit flies were allowed to lay eggs for 8-12 hours. Animals were heat shocked in a 37° C. water bath for 1 h from 0 to 100 h AEL. Brains from adult animals were dissected 3-5 days after eclosion. The genotype of GH146 coupled MARCM flies is: hsFLP, QUAS-mtdT-3xHA, UAS-mCD8-GFP(X); GH146-QF#53, 82B^{FRT}, tub-QS/tubP-GAL4, 82B^{FRT}, tubP-GAL80 (III).

Coupled MARCM analysis of wing disc clones: Vials containing approximately 15 males and 25 females were allowed to lay eggs for 6-8 h. The progeny were heat shocked in a 37° C. water bath for 30 minutes at 48±3 h after egg laying (AEL), and dissected 72±3 h after the heat shock. This 30-minute heat shock leads to about 1-2 wild-type coupled MARCM wing clones per disc. (A 1 h heat shock leads to approximately 5-10 clones per disc.) In control coupled MARCM experiments, coupled MARCM clones in the wing imaginal disc could be visualized in live samples (unfixed/unstained) as early as 28 h after the clone-inducing heat shock (clones were induced by a 2 h heat shock at 60 h AEL). This observation suggests that perdurance of GAL80 and QS is minimal 28 h after clone induction.

Quinic acid feeding of flies: Quinic acid containing medium was made as follows: a few holes were made in standard fly medium with wooden sticks, and 0.3 ml of freshly made D-(−)-quinic acid (Sigma-Aldrich, 98%, Catalog #138622) solution dissolved in water was added per 10 ml of medium. Vials were allowed to air-dry overnight. Quinic acid appears to be stable in food stored for at least a week at 18° C., as judged by its derepression activity (e.g. FIG. 3D). For assaying the effect of quinic acid on developing animals, approximately 10 males and 15 females per vial of the genotype ET40-QF, QUAS-mtdT-3xHA (II); 82B^{FRT} tubP-QS (III) were allowed to lay eggs for 6 hours on quinic acid containing medium. Animals fed upon and developed in the quinic acid containing food. Adult flies were imaged within 3 days of eclosion. For assaying the effect of quinic acid feeding on adult flies, approximately 30 adult flies per vial of genotype ET40-QF, QUAS-mtdT-3xHA (II); 82B^{FRT} tubP-QS (III) were allowed to feed on quinic acid medium for the listed times in FIG. 3D. For feedings over multiple days, flies were transferred to fresh quinic acid containing food every two days. Starving flies for 24 h did not lead to increased suppression, suggesting that both well-fed and hungry flies ingested the quinic acid food at similar levels. Flies fed with quinic acid food for up to 10 days exhibited no more derepression than those fed for 5 days.

Imaging and Image Processing

Immunohistochemistry: Confocal images were taken on a LSM 510 Confocal Microscope (Zeiss). The procedures for fixation, immunochemistry and imaging were as described previously (Wu and Luo, 2006). Primary antibodies used were Rat anti-CD8 (Caltag Laboratories, 1:200), Mouse anti-HA (12CA5, 1:100), Rabbit anti-HA (Abcam, 1:100), Rat anti-DNCadherin (DN-EX#8, DSHB, 1:25), Mouse anti-nc82 (DSHB, 1:25), Rabbit anti-β-galactosidase (1:100), Chicken anti-GFP (Ayes Labs, 1:100), Mouse anti-acj6 (DSHB, 1:50), Mouse anti-fibrillarin (72B9, 1:20), Rat anti-ELAV (7E8A10, DSHB, 1:100), Mouse anti-Repo (8D12, DSHB, 1:50), Mouse anti-24B10 (1:25).

Imaging of adult flies: Adult flies were imaged on a Qlmaging Retiga 2000R Cooled Monochrome Digital camera using a Discovery V8 Pentafluor System (Zeiss) with a DS RED filter cube.

GH146 expression pattern: Both GH146-QF transgenic lines described in this study have approximately equal PNs expression patterns. The major difference is that GH146-QF#11 is expressed in fewer ventral PNs than GH146-QF#53. GH146-QF#53 was used for all GH146 coupled MARCM experiments.

Wing imaginal disc quantification: A customized Matlab script was used for the automated calculation of clone size and cell number in confocal stacks. Cell size (area) was calculated by dividing clone area by cell number. Confocal images were initially processed so that only one set of coupled MARCM clones was present in a confocal stack. The clone area was defined by first thresholding the red channel (mtdT-HA staining) or green channel (mCD8-GFP staining) to delineate the clone, and then calculated by summing the number of pixels above the set threshold. Clone areas in the red and green channels were independently calculated throughout the image stack. The z plane containing the largest clone area and two adjacent planes were recorded. The final clone size, cell number and cell size for each image were the average values of these three z planes. For automated counting of cell numbers in the stack, the thresholded red or green clone areas were used as a mask to define the region of interest in the blue channel (anti-fibrillarin staining which marks the single nucleolus in each cell). The algorithm for counting cell numbers was adapted from the ITCN Matlab script by Thomas Kuo and Jiyun Buyn (Center for Bioimage Informatics). The parameters for automated measurements were set according to test measurements of clone area and cell number, which were obtained by manually counting a small randomly selected image set. The obtained numbers were compared for statistical significance using Student's paired t-test.

Behavioral Analysis

Olfactory attraction was measured using a modified two choice trap assay (Larsson et al., 2004). 2-3 day old adult flies were starved for 40-42 h in collection cages containing water moisted Kimwipes. Approximately 100 flies per assay were anaesthetized by cold and placed into a small culture dish at the bottom of a 85 mm×170 mm, 1000 ml glass jar (Fisher, 02-912-305) covered by a 150×15 mm Petri dish (Falcon, 351058) that had three nylon mesh screened holes inserted for ventilation. Odor traps were constructed from 40 ml glass vials (National Scientific B7999-6) to which a custom-built polyethylene top containing a cut pipette tip was securely placed. Traps contained a cotton foam plug to which either 0.5 ml of 1% ethyl acetate (Sigma, >99.5% purity) dissolved in mineral oil, or mineral oil alone, were added. The behavioral tests were conducted for two hours in a dark humidified room at 34° C.

### EXAMPLES

The following examples are put forth so as to provide those of ordinary skill in the art with a complete disclosure and description of how to make and use the present invention; they are not intended to limit the scope of what the inventors regard as their invention. Unless indicated otherwise, part are parts by weight, molecular weight is average molecular weight, temperature is in degrees Centigrade, and pressure is at or near atmospheric.

### Example 1

### Characterization of the Q-System in Drosophila and Mammalian Cells

To test whether qa cluster genes function in biological systems besides Neurospora, we created expression constructs for transient transfection of Drosophila and mammalian cells. We used the same ubiquitous promoters to drive QF and QS: actin 5c for Drosophila and CMV for mammalian cells. We generated a reporter plasmid containing the synthetic firefly luciferase (luc2) gene under the control of 5 copies of the QF binding site, which we termed QUAS, and the Drosophila hsp70 minimal promoter. We also created the GAL4-system equivalents as controls and for quantitative comparisons with the Q system.

Transfection of Drosophila S2 cells with QF and QUAS-luc2 resulted in ˜3.300-fold enhancement of luc2 expression compared with QUAS-luc2 alone (FIG. 1B). For comparison, GAL4 induced luc2 expression from UAS-luc2 by ˜5,300-fold (FIG. 1B) and therefore had ˜1.6-fold higher inducibility than QF/QUAS. GAL4/UAS also reached ˜1.8-fold higher absolute level of reporter expression than QF/QUAS. Co-transfection of QS with QF and QUAS-luc2 resulted in dosage-dependent suppression of luc2-expression (FIG. 1B). Full suppression was not observed with equimolar ratios of QF and QS (similar lack of full suppression was observed with GAL4/GAL80). Quinic acid, which relieves suppression of QS in Neurospora (Giles et al., 1991), significantly suppressed QS to restore QF-based transcription (FIG. 2A). Finally, QF and GAL4 showed minimal cross-activation of UAS and QUAS, respectively (FIG. 1B, middle)—QF activation of UAS was ˜1,500 fold less than that of QUAS; GAL4 activation of QUAS was ˜200 fold less than that of UAS.

In human HeLa cells (FIG. 1C), the Q system behaved similarly as in Drosophila S2 cells, but with the following distinctions. First, QF induced expression from QUAS by ˜24.000-fold, compared to ˜1000-fold induction of UAS by GAL4. Therefore, in human cells, QF/QUAS achieves ˜24-fold higher inducibility and ˜30-fold higher absolute level of reporter expression than GAL4/UAS. Second, higher QS:QF or GAL80:GAL4 molar ratios are required for effective suppression in HeLa cells compared with Drosophila S2 cells. Third, quinic acid does not suppress QS in mammalian cells, but seems to activate it further to make it an even better repressor (FIG. 2B); the reasons for this unexpected behavior in mammalian cells are unknown. All these distinctions were also observed in COS cells (data not shown). Taken together, these experiments demonstrate that the Q repressible binary expression system is effective in Drosophila and mammalian cells.

### Example 2

### In-Vivo Characterization of the Q-System in Flies

Repressible Binary Transgene Expression Using the Q System in Drosophila

To test whether the Q system functions in Drosophila in vivo, we generated transgenic flies that express: 1) different markers under the control of QUAS, 2) QF under the control of a specific promoter or in enhancer trap vectors, and 3) QS under the control of a ubiquitous tubulin promoter (tubP-QS) (Table 1).

FIG. 2A-B (left panels) shows low basal fluorescence in whole mount Drosophila adult brains harboring only reporter transgenes, QUAS-mCD8-GFP (full length mouse CD8 followed by GFP) or QUAS-mtdT-HA (myristoylated and palmitoylated tandem repeat Tomato followed by 3 copies of the HA epitope). The low basal expression of QUAS and UAS reporters provides significant advantage compared to the lexA binary expression system (Lai and Lee, 2006). All QUAS-mCD8-GFP transgenic flies have basal reporter expression comparable to or lower than the lexO-mCD2-GFP line with the lowest reporter expression (FIG. 4A). Low basal expression was also observed in other QUAS reporters such as QUAS-mdtT-HA (FIG. 4A, data not shown). These observations suggest that the QUAS promoter is not easily influenced by genomic enhancers near the transgene insertion site and that flies do not contain endogenous proteins capable of inducing significant expression from QUAS-transgenes at least within the tissues we examined.

Introduction of QF expressing transgenes into flies containing QUAS-markers results in strong marker expression. For example, QF driven by the GH146 enhancer (Stocker et al., 1997; Berdnik et al., 2008) drives strong transgene expression in olfactory projection neurons (PNs; FIGS. 2A2-3 and 2B2-3). We also isolated enhancer trap lines that drive strong reporter expression in imaginal discs and adult tissues including large subsets of neurons and glia (FIG. 2C, middle; FIG. 4B, S3). Expression of these transgenes was effectively suppressed by ubiquitous expression of QS (FIGS. 2A4, 2B4 and 2C, right; FIG. 4B). These experiments show that the Q repressible binary system is as effective in vivo as the widely used GAL80/GAL4/UAS system (Brand and Perrimon, 1993; Lee and Luo, 1999).

The Q system provides an additional level of control compared to the GAL4 system: inhibition of QS by quinic acid. Adding increasing doses of quinic acid to fly food on which flies developed increasingly reverted the QS inhibition of enhancer trap ET40-QF driven QUAS-mtdT-HA expression (FIG. 2D). When adult flies were transferred to quinic acid-containing food, reversion of suppression could be seen after 6 h, with marked reversion after 24 h and saturation by day 5 (FIG. 2E, data not shown). Flies kept for 9 generations on food containing high doses of quinic acid, a natural product present at >1% in cranberry juice (Nollet, 2000), exhibited no noticeable abnormalities. Quinic acid can thus be used to temporally regulate QF-driven transgene expression. For instance, one can suppress developmental expression of a transgene and allow reactivation in adult for behavioral analysis, analogously to the GAL80ts strategy (McGuire et al., 2003). This manipulation can be achieved without changing the temperature, thereby avoiding complications with temperature-sensitive behaviors.

### Example 3

### Development of GAL4-Independent MARCM System

Q-MARCM

An incentive to develop the Q repressible binary system is the potential to build a new GAL4-independent MARCM system. The Q system-based MARCM (Q-MARCM) can then be used to mark and genetically manipulate a single cell or a small population of cells, while GAL4/UAS can be used to genetically manipulate a separate population of cells in the same animal. To test Q-MARCM, we placed tubP-QS distally to an FRT site and used FLP/FRT to induce mitotic recombination, so that one of the two daughter cells would lose tubP-QS, thus permitting QF to drive QUAS-marker expression (FIG. 6A).

Using GH146-QF to label olfactory PNs in Q-MARCM experiments, we found single cell and neuroblast clones labeled by QUAS-mCD8-GFP (FIG. 6B) or QUAS-mtdT-HA (see below). In single cell clones, the dendritic innervation of individual glomeruli in the antennal lobe and stereotyped projections of single axons in the lateral horn appeared indistinguishable from previously characterized single cell clones labeled by GH146-GAL4-based MARCM (Jefferis et al., 2001; Marin et al., 2002; Jefferis et al., 2007). We have validated tubP-QS transgenes on all five major chromosome arms (Table 1), thereby allowing GAL4-independent MARCM analysis for a vast majority of Drosophila genes using the Q system.

GAL4 and QF showed minimal cross-activation of their respective upstream activating sequences in cultured cells (FIG. 1B, C). Moreover, we could not detect any cross-activation (FIG. 7A) or cross-repression (FIG. 7B) of the GAL4 and QF systems in vivo. Therefore, QF- and GAL4-based MARCM (G-MARCM) can be combined in the same fly. If tubP-GAL80 and tubP-QS are placed distally to FRT sites on different chromosome arms (FIG. 7C), independently generated clones can be labeled by Q- and G-MARCM. This arrangement, which we term ‘independent double MARCM’, can be used to study interactions between two separate populations of cells that have undergone independent mitotic recombination and genetic alteration. If tubP-GAL80 and tubP-QS transgenes are placed distally to the same FRT site in trans (FIG. 6C), sister cells resulting from the same mitotic recombination can be labeled by Q- and G-MARCM respectively. We call the latter case ‘coupled MARCM’.

FIG. 6D illustrates an example of coupled MARCM in the third instar larval eye disc. Sister cells and their descendants, derived from a single mitotic recombination event based on clone frequency and the proximity of labeled cells, are marked by tubP-GAL4 driven UAS-mCD8-GFP and ET40-QF driven QUAS-mtdT-HA. The photoreceptor cell bodies and their axonal projections into the brain were clearly visualized by both G-MARCM and Q-MARCM.

### Example 4

### Analysis of Lineage and Cell Division Patterns Using Coupled MARCM

The ability to label both progeny of a dividing cell with different colors via coupled MARCM (FIG. 6C) can be used to characterize two important aspects of a developmental process: cell lineage and division patterns. As an example to illustrate such utility, we investigated the cell division pattern of a central nervous system neuroblast that gives rise to the adult olfactory PNs.

The cell division patterns of neuroblasts that generate adult insect CNS neurons are thought to follow the scheme shown in FIG. 8A: a neuroblast undergoes asymmetric divisions to produce a new neuroblast and a ganglion mother cell (GMC), which divides once more to produce two postmitotic neurons (Nordlander and Edwards, 1969). A previous GAL4-based MARCM analysis of the mushroom body lineage supports this model: neuroblast, two-cell and single-cell clones can be produced (FIG. 8B), and the frequency of the neuroblast and two-cell clones are roughly equal, reflecting the random segregation of the GAL80-containing chromosomes into the neuroblast or the GMC (Lee et al., 1999; Lee and Luo, 1999). However, when we analyzed PN lineages using MARCM and GH146-GAL4 (Jefferis et al., 2001) or GH146-QF (data not shown), we obtained either neuroblast or single cell clones, but no two-cell PN clones. Three different models can account for these data (FIG. 8C). In model I, the stereotypical division pattern (FIG. 8A) does not apply to this lineage: GH146-positive PNs are direct descendants of the neuroblasts. In models II and III, the general division pattern still applies, but the sibling for the GH146-positive PN is either a GH146-negative cell (model II), or it dies (model III).

We used coupled MARCM to distinguish among these models, focusing on the best-characterized anterodorsal lineage in which all progeny are PNs (Lai et al., 2008) and where birth order has been determined for most GH146-positive PNs (Jefferis et al., 2001; Marin et al., 2005). We used GH146-QF to label PNs derived from one progeny of a cell division, and the ubiquitous tubP-GAL4 to label the sibling progeny (FIG. 9). We induced clones by heat-shock at different time windows within 0-100 h after egg laying and recovered a total of 91 coupled MARCM clones. We sorted the clones according to their labeling by GH146-QF and tubP-GAL4 (FIG. 8D).

If model I were true, a single PN should always have a neuroblast sibling (FIG. 8C1). However, we found 19 out of 44 single PNs labeled by GH146-QF without a tubP-GAL4 labeled neuroblast clone (FIG. 8E1), and 5 out of 38 single PNs labeled by tubP-GAL4 without a GH146-QF labeled neuroblast clone (FIG. 8E2). Thus, model I does not apply.

If model II were true, GH146-QF labeled neuroblast clones should be coupled with a two-cell clone labeled by the ubiquitous tubP-GAL4 (regardless of them being GH146-positive or GH146-negative; FIG. 8C2 left). However, of the 40 GH146-QF labeled neuroblast clones, none of the tubP-GAL4 siblings were two-cell clones (FIG. 8D). Instead, in 33 cases, the siblings were single cell clones (FIG. 8E3), and in the other 7 cases, there were no labeled siblings (FIG. 8E4). In addition, model II would predict pairs of sister cells each labeled by tubP-GAL4 or GH146-QF as a result of mitotic recombination in the GMC (FIG. 8C2, right), but such an event was never observed (FIG. 8D).

These experiments therefore support model III: the sibling of each PN dies during development and is no longer present in the adult brain (FIG. 8C3). The frequent occurrence of single singly-labeled PNs without labeled siblings could result from mitotic recombination in the GMC giving rise to two cells, one of which dies (bottoms of FIG. 8E1, 8E2). In addition, occasionally both GMC-derived siblings may die, giving rise to neuroblast clones without any labeled siblings (FIG. 8E4). This model is also supported by a recent study using different methods (Lin et al., 2010). It is possible that the division patterns producing PNs vary at different developmental stages and for different lineages.

### Example 5

### Analyzing Cell Proliferation and Growth Using Coupled MARCM

Coupled MARCM allows direct comparison of two cell populations that arise from a single cell division within the same animal. Here we illustrate its use to study cell proliferation and growth in the wing imaginal disc (FIG. 10A).

The ˜50,000 epithelial cells of the wing disc are produced by exponential cell division from less than 40 progenitor cells during the larval stages of Drosophila development (Bryant and Simpson, 1984). Clonal analysis in the wing imaginal disc is a sensitive strategy for studying the effects of genetic perturbations on cell growth or proliferation. To verify that QF expression does not affect normal cell growth or proliferation, we used coupled MARCM to label wild-type clones in the larval wing imaginal disc (FIG. 10B). Clones were induced by heat-shock at 48 h after egg laying, and examined 72 h later. The area of the GAL4- and QF-labeled clones, their cell number and cell size (FIGS. 10D, 10E and 10F, respectively) were indistinguishable from one another. These results indicate that G-MARCM and Q-MARCM do not differentially affect cell proliferation or growth of wing disc cells. Additional control experiments indicated that high levels of QF expression did not interfere with growth and patterning of imaginal discs and the corresponding adult structures (FIG. 11).

To show the utility of coupled MARCM in mutant analysis, we generated wing imaginal disc clones in which control cells were labeled by GAL4 and Tuberous Sclerosis 1 (Tsc1) homozygous mutant cells were labeled by QF. Tsc1, along with its partner Tuberous Sclerosis 2 (Tsc2), forms a complex that negatively regulates the Tor pathway to affect both cell size and cell proliferation (Ito and Rubin, 1999; Potter et al., 2001; Tapon et al., 2001). We found that Tsc1 mutant clones (labeled red via QF) were significantly larger than wild-type clones (labeled green via GAL4) (FIG. 10C), covering on average 2.9-fold larger area than their control sister clones (FIG. 10D). To determine if the increase in clone area is due to an increase in cell proliferation or cell size, we counted the number of cells within these labeled clones. We found a two-fold increase in cell numbers in Tsc1 mutant clones compared to the sister clones, yet only a 26% increase in cell size (FIG. 10E-F), suggesting that mutation of Tsc1 in rapidly dividing cells primarily leads to an increase in proliferative capacity. This example, although largely confirmatory of previous findings, illustrates the utility of coupled MARCM for investigating gene function in developmental processes.

### Example 6

### Refining Transgene Expression by Intersecting GAL4 and QF Expression Patterns

A major power of the GAL4/UAS system is its ability to manipulate many cell types through thousands of GAL4 lines generated by enhancer trapping or GAL4 fusions to specific promoters. Despite the abundance of GAL4 lines, their expression patterns are often too broad to establish the causality between the expression of a transgene in a particular cell type and a phenotype, especially if the phenotype is assayed at the organismal level. Combining GAL4- and QF-based binary systems into logic gates can create new expression patterns (FIG. 13). Below we provide proof-of-principle examples for some of these strategies (FIG. 12).

QF NOT GAL4. Like the previously characterized GH146-GAL4 (Jefferis et al., 2001), GH146-QF is expressed in PNs that are derived from the anterodorsal, lateral and ventral neuroblast lineages (FIG. 2B). The POU transcription factor Acj6 is expressed only in anterodorsal but not in lateral or ventral GH146-positive PNs (Komiyama et al., 2003). Acj6, and acj6-GAL4, an enhancer trap line inserted into the acj6 locus, are also expressed in some GH146-negative anterodorsal PNs, in many ORNs, in atypical PNs, and in lateral horn output neurons (Clyne et al., 1999; Komiyama et al., 2003; Suster et al., 2003; Komiyama et al., 2004; Jefferis et al., 2007; Lai et al., 2008). As shown in FIG. 12A, when GH146-QF and acj6-GAL4 are present in the same fly, and are detected via QUAS-mtdT-HA and UAS-mCD8-GFP, respectively, a large subset of anterodorsal PNs is labeled by both mCD8-GFP and mtdT-HA, whereas lateral and ventral PNs express mtdT-HA but not mCD8-GFP.

By introducing a UAS-QS transgene, we subtracted the GAL4-expressing cells from the QF-expressing cells such that the QUAS-mtdT-HA reporter was only expressed in the lateral and ventral, but not the anterodorsal PNs (FIG. 12B; compare FIG. 12B3 with 12A3). In this manner, we created ‘QF NOT GAL4’, a new QF-dependent expression pattern. Using this logic gate, we observed non-overlapping glomeruli labeled by Acj6-expressing anterodorsal PNs in green and QF-expressing lateral PNs in red (FIG. 12B2). This observation confirms directly in the same animal a previous finding that PNs from the anterodorsal and lateral lineages project dendrites to complementary and non-overlapping glomeruli in the antennal lobe (Jefferis et al., 2001).

Expression pattern subtraction can also be visualized at the level of axon terminals. Both anterodorsal and lateral PNs project their axonal collaterals into the mushroom body calyx, where they terminate in large presynaptic boutons. In the absence of the UAS-QS transgene, these individual terminal boutons are labeled green, yellow and red, representing axon terminals of PNs that are Acj6+/GH146− anterodorsal PNs, Acj6+/GH146+ anterodorsal PNs, and GH146+/Acj6− lateral PNs, respectively (FIG. 12A4). In the presence of UAS-QS, yellow terminal boutons are no longer present (FIG. 12B4), indicating that acj6-GAL4 labeled cells have been subtracted from the GH146-QF expression pattern. This experiment allows, for the first time, a direct comparison of axon terminal distributions of anterodorsal and lateral PNs co-innervating the same mushroom body.

QF AND GAL4. By introducing two additional transgenes, QUAS-FLP, UAS>stop>effector (FIG. 12C1, 12D1; > represents FRT), or UAS-FLP, QUAS>stop>effector (FIG. 12E1), into an animal containing a GAL4 and a QF line, only cells that express both QF and GAL4 ('QF AND GAL4′) can be selectively visualized and genetically manipulated. Below we show three examples.

First, we studied the intersection of GH146-QF and acj6-GAL4. With the introduction of UAS-FLP and QUAS>stop>mCD8-GFP, anterodorsal PNs that are both Acj6+ and GH146+ were labeled (FIG. 12C), confirmed by the glomerular identity of dendritic projections of these neurons (data not shown). A previously described Acj6/GH146 double-positive cell from a separate lineage (Komiyama et al., 2003) was also labeled (FIG. 12C4, arrowhead). All other lateral and all ventral GH146+PNs, which do not express Acj6, no longer expressed the marker. The marker was also not expressed in ORNs or lateral horn neurons, which express Acj6 but not GH146. Thus, we can express transgenes only in cells that express both GH146 and Acj6: a subset of anterodorsal PNs.

In the second and third examples, we studied the intersection between GH146-QF and NP21-GAL4 using two AND gate strategies. NP21-GAL4 is an enhancer trap line inserted near the promoter of fruitless (fru) (Hayashi et al., 2002) that drives the expression of the male-specific isoform of Fru (FruM), which is essential for regulating mating behavior (Demir and Dickson, 2005; Manoli et al., 2005). NP21-GAL4 labels many neurons in the brain (Kimura et al., 2005)(FIG. 12D2), including PNs that project dendrites to the DA1 glomerulus (FIG. 12E2). In our first strategy (FIG. 12D1), we used UAS-FLP and QUAS>stop> mCD8-GFP, and found that ˜10 PNs that innervated several glomeruli were selectively labeled (FIG. 12D3, 12D4). In our second strategy (FIG. 12E1), we used QUAS-FLP and UAS>stop>mCD8-GFP, and found that the labeled PNs were restricted to only ˜5 cells that project their dendrites to the DA1 glomerulus (FIG. 12E3, 12E4). The difference between these two strategies reflects the fact that in these intersectional strategies, the binary system used to drive FLP reports the cumulative developmental history, rather than only the adult expression, of the driver. Our data suggest that NP21-GAL4 (and by inference fruM) is expressed in more PN classes during development than in the adult. In both cases, the complex NP21-GAL4 expression pattern outside of PNs has been reduced to very few cells. The comparison of expression patterns from the two strategies can pinpoint the cells that are at the intersection of GH146-QF and NP21-GAL4 adult expression patterns.

Comparisons with other methods. An AND gate can be achieved by utilizing the split-GAL4 system (Luan et al., 2006). The benefit of our method is that it can take advantage of the thousands of available and well-characterized GAL4 lines, whereas the split-GAL4 system needs to generate new split N-GAL4 and C-GAL4 lines. In addition, reconstituted GAL4 from the split GAL4 system is not as strong as wild-type GAL4 in driving transgene expression(Luan et al., 2006).

The intersection between FLP/FRT and GAL4/UAS can also be used directly as an AND gate without going through a second binary system to express FLP (Stockinger et al., 2005; Hong et al., 2009). Both this method and our method have the caveats of transient FLP expression during development, as well as the possibility that FLP/FRT-mediated recombination may not occur in all cells that express FLP. Although our method requires one additional transgene, it offers several advantages over promoter-driven FLP. First, our method does not require the generation of separate tissue or cell type-specific FLP lines. Second, by inducing higher FLP levels due to transcriptional amplification of binary expression, our method should more readily overcome problems of incomplete recombination. Indeed, counts of the number of DA1 projecting PNs that are part of the NP21 expression pattern with or without the AND gate with GH146 are similar (NP21-GAL4: 5.2±0.1, n=48; GH146-QF/QUAS-FLP AND NP21-GAL4: 5.1±0.1, n=10), suggesting nearly complete FLP/FRT mediated recombination. Third, our method offers two complementary AND gate strategies, which together can be used to overcome the ambiguities arising from transient developmental expression. Fourth, transient developmental expression mediated by QUAS-FLP could in principle be suppressed by introducing tubP-QS, and the suppression could be reversed by supplying the flies with quinic acid at appropriate developmental stages.

The ‘QF NOT GAL4’ or ‘GAL4 NOT QF’ (FIG. 13) strategies are conceptually similar to GAL80 subtraction of GAL4 expression (Lee and Luo, 1999). If one were to generate a large number of GAL80 enhancer trap or promoter driven lines, one could use this set to subtract their expression patterns from GAL4 expression patterns. One limitation of this approach is that the GAL80 expression pattern is difficult to determine at high resolution because it is based on suppression of GAL4-induced gene expression. In addition, GAL80 levels must be sufficiently high to ensure proper suppression of GAL4, which may not be true for many enhancer trap or promoter-driven GAL80 transgenes. By contrast, the “NOT” gate we describe here utilizes the expression patterns of two transcription factors, which express the appropriate repressor through binary amplification, and should therefore circumvent both limitations above.

A major limitation of our intersectional strategies for refinement of gene expression is the availability of QF drivers with different expression patterns. So far, we were unsuccessful in generating tubP-QF transgenic animals, suggesting that QF is toxic to flies when highly expressed in a ubiquitous manner or in a particular developmental stage or tissue (see experimental procedures, infra). Nonetheless, we isolated many QF enhancer traps that express strongly in imaginal discs, epithelial tissues, glia, and neurons (FIG. 4B, FIG. 5). We hope that our proof-of-principle examples here will stimulate the Drosophila community to generate large numbers of enhancer trap and promoter-driven QF lines in the future. The number of new expression patterns created by intersections between GAL4 and QF should be multiplicative. For instance, 100 QF lines in combination with 10,000 GAL4 lines, given sufficient expression overlap and utilizing different logic gates (FIG. 13), should in principle generate millions of new effector expression patterns.

Although the foregoing invention and its embodiments have been described in some detail by way of illustration and example for purposes of clarity of understanding, it is readily apparent to those of ordinary skill in the art in light of the teachings of this invention that certain changes and modifications may be made thereto without departing from the spirit or scope of the appended claims. Accordingly, the preceding merely illustrates the principles of the invention. It will be appreciated that those skilled in the art will be able to devise various arrangements which, although not explicitly described or shown herein, embody the principles of the invention and are included within its spirit and scope.

### REFERENCES

Baum, J. A., Geever, R., and Giles, N. H. (1987). Expression of qa-1F activator protein: identification of upstream binding sites in the qa gene cluster and localization of the DNA-binding domain. Mol Cell Biol 7, 1256-1266.
Berdnik, D., Chihara, T., Couto, A., and Luo, L. (2006). Wiring stability of the adult Drosophila olfactory circuit after lesion. J Neurosci 26, 3367-3376.
Berdnik, D., Fan, A. P., Potter, C. J., and Luo, L. (2008). MicroRNA processing pathway regulates olfactory neuron morphogenesis. Curr Biol 18, 1754-1759.
Bischof, J., Maeda, R. K., Hediger, M., Karch, F., and Basler, K. (2007). An optimized transgenesis system for Drosophila using germ-line-specific phiC31 integrases. Proc Natl Acad Sci USA 104, 3312-3317.
Brand, A. H., and Perrimon, N. (1993). Targeted gene expression as a means of altering cell fates and generating dominant phenotypes. Development 118, 401-415.
Bryant, P. J., and Simpson, P. (1984). Intrinsic and extrinsic control of growth in developing organs. Q Rev Biol 59, 387-415.
Clyne, P. J., Certel, S. J., de Bruyne, M., Zaslaysky, L., Johnson, W. A., and Carlson, J. R. (1999). The odor specificities of a subset of olfactory receptor neurons are governed by Acj6, a POU-domain transcription factor. Neuron 22, 339-347.
Demir, E., and Dickson, B. J. (2005). fruitless splicing specifies male courtship behavior in Drosophila. Cell 121, 785-794.
Dietzl, G., Chen, D., Schnorrer, F., Su, K. C., Barinova, Y., Fellner, M., Gasser, B., Kinsey, K., Oppel, S., Scheiblauer, S., et al. (2007). A genome-wide transgenic RNAi library for conditional gene inactivation in Drosophila. Nature 448, 151-156.
Fischer, J. A., Giniger, E., Maniatis, T., and Ptashne, M. (1988). GAL4 activates transcription in Drosophila. Nature 332, 853-856.
Gerlitz, O., Nellen, D., Ottiger, M., and Basler, K. (2002). A screen for genes expressed in Drosophila imaginal discs. Int J Dev Biol 46, 173-176.
Giles, N. H., Geever, R. F., Asch, D. K., Avalos, J., and Case, M. E. (1991). The Wilhelmine E. Key 1989 invitational lecture. Organization and regulation of the qa (quinic acid) genes in Neurospora crassa and other fungi. J Hered 82, 1-7.
Golic, K. G., and Lindquist, S. (1989). The FLP recombinase of yeast catalyzes site-specific recombination in the Drosophila genome. Cell 59, 499-509.
Gossen, M., and Bujard, H. (1992). Tight control of gene expression in mammalian cells by tetracycline-responsive promoters. Proc Natl Acad Sci USA 89, 5547-5551.
Griffin, R., Sustar, A., Bonvin, M., Binari, R., del Valle Rodriguez, A., Hohl, A. M., Bateman, J. R., Villalta, C., Heffern, E., Grunwald, D., et al. (2009). The twin spot generator for differential Drosophila lineage analysis. Nat Methods 6, 600-602.
Groth, A. C., Fish, M., Nusse, R., and Calos, M. P. (2004). Construction of transgenic Drosophila by using the site-specific integrase from phage phiC31. Genetics 166, 1775-1782.
Hayashi, S., Ito, K., Sado, Y., Taniguchi, M., Akimoto, A., Takeuchi, H., Aigaki, T., Matsuzaki, F., Nakagoshi, H., Tanimura, T., et al. (2002). GETDB, a database compiling expression patterns and molecular locations of a collection of Gal4 enhancer traps. Genesis 34, 58-61.
Hong, W., Zhu, H., Potter, C. J., Barsh, G., Kurusu, M., Zinn, K., and Luo, L. (2009). Leucine-rich repeat transmembrane proteins instruct discrete dendrite targeting in an olfactory map. Nat Neurosci 12, 1542-1550.
Huiet, L., and Giles, N. H. (1986). The qa repressor gene of Neurospora crassa: wild-type and mutant nucleotide sequences. Proc Natl Acad Sci USA 83, 3381-3385.
Ito, N., and Rubin, G. M. (1999). gigas, a Drosophila homolog of tuberous sclerosis gene product-2, regulates the cell cycle. Cell 96, 529-539.
Jefferis, G. S., Potter, C. J., Chan, A. M., Marin, E. C., Rohlfing, T., Maurer, C. R., Jr., and Luo, L. (2007). Comprehensive maps of Drosophila higher olfactory centers: spatially segregated fruit and pheromone representation. Cell 128, 1187-1203.
Jefferis, G. S. X. E., Marin, E. C., Stocker, R. F., and Luo, L. (2001). Target neuron prespecification in the olfactory map of Drosophila. Nature 414, 204-208.
Kimura, K., Ote, M., Tazawa, T., and Yamamoto, D. (2005). Fruitless specifies sexually dimorphic neural circuitry in the Drosophila brain. Nature 438, 229-233.
Kitamoto, T. (2001). Conditional modification of behavior in Drosophila by targeted expression of a temperature-sensitive shibire allele in defined neurons. J Neurobiol 47, 81-92.
Komiyama, T., Carlson, J. R., and Luo, L. (2004). Olfactory receptor neuron axon targeting: intrinsic transcriptional control and hierarchical interactions. Nat Neurosci 7, 819-825.
Komiyama, T., Johnson, W. A., Luo, L., and Jefferis, G. S. (2003). From lineage to wiring specificity: POU domain transcription factors control precise connections of Drosophila olfactory projection neurons. Cell 112, 157-167.
Lai, S. L., Awasaki, T., Ito, K., and Lee, T. (2008). Clonal analysis of Drosophila antennal lobe neurons: diverse neuronal architectures in the lateral neuroblast lineage. Development 135, 2883-2893.
Lai, S. L., and Lee, T. (2006). Genetic mosaic with dual binary transcriptional systems in Drosophila. Nat Neurosci 9, 703-709.
Larsson, M. C., Domingos, A. I., Jones, W. D., Chiappe, M. E., Amrein, H., and Vosshall, L. B. (2004). Or83b encodes a broadly expressed odorant receptor essential for Drosophila olfaction. Neuron 43, 703-714.
Lee, T., Lee, A., and Luo, L. (1999). Development of the Drosophila mushroom bodies: sequential generation of three distinct types of neurons from a neuroblast. Development 126, 4065-4076.
Lee, T., and Luo, L. (1999). Mosaic analysis with a repressible cell marker for studies of gene function in neuronal morphogenesis. Neuron 22, 451-461.
Lin, S., Lai, S. L., Yu, H. H., Chihara, T., Luo, L., and Lee, T. (2010). Lineage-specific effects of Notch/Numb signaling in post-embryonic development of the Drosophila brain. Development 137, 43-51.
Luan, H., Peabody, N. C., Vinson, C. R., and White, B. H. (2006). Refined spatial manipulation of neuronal function by combinatorial restriction of transgene expression. Neuron 52, 425-436.
Luo, L. (2007). Fly MARCM and mouse MADM: genetic methods of labeling and manipulating single neurons. Brain Res Rev 55, 220-227.
Luo, L., Callaway, E. M., and Svoboda, K. (2008). Genetic dissection of neural circuits. Neuron 57, 634-660.
Manoli, D. S., Foss, M., Villella, A., Taylor, B. J., Hall, J. C., and Baker, B. S. (2005). Male-specific fruitless specifies the neural substrates of Drosophila courtship behaviour. Nature 436, 395-400.
Marin, E. C., Jefferis, G. S. X. E., Komiyama, T., Zhu, H., and Luo, L. (2002). Representation of the glomerular olfactory map in the Drosophila brain. Cell 109, 243-255.
Marin, E. C., Watts, R. J., Tanaka, N. K., Ito, K., and Luo, L. (2005). Developmentally programmed remodeling of the Drosophila olfactory circuit. Development 132, 725-737.
Markstein, M., Pitsouli, C., Villalta, C., Celniker, S. E., and Perrimon, N. (2008). Exploiting position effects and the gypsy retrovirus insulator to engineer precisely expressed transgenes. Nat Genet. 40, 476-483.
McGuire, S. E., Le, P. T., Osborn, A. J., Matsumoto, K., and Davis, R. L. (2003). Spatiotemporal rescue of memory dysfunction in Drosophila. Science 302, 1765-1768.
Muzumdar, M. D., Tasic, B., Miyamichi, K., Li, L., and Luo, L. (2007). A global double-fluorescent Cre reporter mouse. Genesis 45, 593-605.
Nollet, L. M. L. (2000). Food analysis by HLPC, 2nd edn (CRC Press).
Nordlander, R. H., and Edwards, J. S. (1969). Postembryonic brain development in the monarch butterfly, Danaus plexippus pleisippus, L. I. cellular events during brain morphogenesis. Wilhelm Roux' rchiv 162, 197-217.
Oberstein, A., Pare, A., Kaplan, L., and Small, S. (2005). Site-specific transgenesis by Cre-mediated recombination in Drosophila. Nat Methods 2, 583-585.
Ornitz, D. M., Moreadith, R. W., and Leder, P. (1991). Binary system for regulating transgene expression in mice: targeting int-2 gene expression with yeast GAL4/UAS control elements. Proc. Natl. Acad. Sci. U.S.A. 88, 698-702.
Patel, V. B., Schweizer, M., Dykstra, C. C., Kushner, S. R., and Giles, N. H. (1981). Genetic organization and transcriptional regulation in the qa gene cluster of Neurospora crassa. Proc Natl Acad Sci USA 78, 5783-5787.
Pfeiffer, B. D., Jenett, A., Hammonds, A. S., Ngo, T. T., Misra, S., Murphy, C., Scully, A., Carlson, J. W., Wan, K. H., Layerty, T. R., et al. (2008). Tools for neuroanatomy and neurogenetics in Drosophila. Proc Natl Acad Sci USA 105, 9715-9720.
Potter, C. J., Huang, H., and Xu, T. (2001). Drosophila Tsc1 functions with Tsc2 to antagonize insulin signaling in regulating cell growth, cell proliferation, and organ size. Cell 105, 357-368.
Raymond, C. S., and Soriano, P. (2007). High-efficiency FLP and PhiC31 site-specific recombination in mammalian cells. PLoS One 2, e162.
Rorth, P., Szabo, K., Bailey, A., Layerty, T., Rehm, J., Rubin, G. M., Weigmann, K., Milan, M., Benes, V., Ansorge, W., and Cohen, S. M. (1998). Systematic gain-of-function genetics in Drosophila. Development 125, 1049-1057.
Rowitch, D. H., B, S. J., Lee, S. M., Flax, J. D., Snyder, E. Y., and McMahon, A. P. (1999). Sonic hedgehog regulates proliferation and inhibits differentiation of CNS precursor cells. J Neurosci 19, 8954-8965.
Spradling, A. C., and Rubin, G. M. (1982). Transposition of cloned P elements into Drosophila germ line chromosomes. Science 218, 341-347.
Stocker, R. F., Heimbeck, G., Gendre, N., and de Belle, J. S. (1997). Neuroblast ablation in Drosophila P[GAL4] lines reveals origins of olfactory interneurons. J Neurobiol 32, 443-456.
Stockinger, P., Kvitsiani, D., Rotkopf, S., Tirian, L., and Dickson, B. J. (2005). Neural circuitry that governs Drosophila male courtship behavior. Cell 121, 795-807.
Suster, M. L., Martin, J. R., Sung, C., and Robinow, S. (2003). Targeted expression of tetanus toxin reveals sets of neurons involved in larval locomotion in Drosophila. J Neurobiol 55, 233-246.
Tapon, N., Ito, N., Dickson, B. J., Treisman, J. E., and Hariharan, I. K. (2001). The Drosophila tuberous sclerosis complex gene homologs restrict cell growth and cell proliferation. Cell 105, 345-355.
Wong, A. M., Wang, J. W., and Axel, R. (2002). Spatial representation of the glomerular map in the Drosophila protocerebrum. Cell 109, 229-241.
Wu, J. S., and Luo, L. (2006). A protocol for dissecting Drosophila melanogaster brains for live imaging or immunostaining. Nat Protoc 1, 2110-2115.
Xu, T., and Rubin, G. M. (1993). Analysis of genetic mosaics in developing and adult Drosophila tissues. Development 117, 1223-1237.
Yu, H. H., Chen, C. H., Shi, L., Huang, Y., and Lee, T. (2009). Twin-spot MARCM to reveal the developmental origin and identity of neurons. Nat Neurosci 12, 947-953.
Zong, H., Espinosa, J. S., Su, H. H., Muzumdar, M. D., and Luo, L. (2005). Mosaic analysis with double markers in mice. Cell 121, 479-492.

What is claimed is:

1) An inducible binary expression system based on regulatory genes from Neurospora crassa, comprising
a) a transcription factor, comprising a DNA binding domain and a transactivation domain;
b) a DNA sequence to which said transcription factor binds;
c) a repressor of said transcription factor; and
d) an effector transgene, whose expression is regulated by the binding of said transcription factor to said DNA sequence.

2) The system of claim 1, further comprising a repressor of said repressor of said transcription factor.

3) The system of claim 2, wherein said repressor of said repressor of said transcription factor is a small molecule, hormone, hormone analog, protein, peptide or metal ion.

4) The system of claim 3, wherein said small molecule is quinic acid.

5) The system of claim 1, wherein said transcription factor is QA-1F (‘QF’).

6) The system of claim 1, wherein said DNA sequence is QUAS.

7) The system of claim 1, wherein said repressor of said transcription factor is QA-1S (‘QS’).

8) A method for controlling timing and level of transgene expression in cells in vitro, the method comprising
a) transforming said cells with a construct comprising a transcription factor comprising a DNA binding domain and a transactivation domain;
b) transforming said cells with a construct comprising a DNA sequence, said transcription factor binding to said DNA sequence;
c) transforming said cells with a construct comprising a repressor of said transcription factor;
d) transforming said cells with an effector transgene, whereby expression of said effector transgene is regulated by binding of said transcription factor.

9) The method of claim 8, further comprising transforming said cells with a repressor of said repressor of said transcription factor.

10) The method of claim 9, wherein said repressor of said repressor of said transcription factor is a small molecule, hormone, hormone analog, protein, peptide or metal ion.

11) The method of claim 10, wherein said small molecule is quinic acid.

12) The method of claim 8, wherein said transcription factor is QA-1F (‘QF’).

13) The method of claim 8, wherein said DNA sequence is QUAS.

14) The method of claim 8, wherein said repressor of said transcription factor is QA-1S (‘QS’).

15) A method for controlling timing and level of transgene expression in an organism in vivo, the method comprising
crossing a first transgenic mouse comprising an effector transgene with a second transgenic mouse comprising a transcription factor, said transcription factor comprising
a DNA binding domain and a transactivation domain;
a DNA sequence for binding of said transcription factor; and
and a repressor of said transcription factor.

16) The method of claim 15, wherein said first or said second transgenic mouse comprises a repressor of said repressor of said transcription factor.

17) The method of claim 16, wherein said repressor of said repressor of said transcription factor is a small molecule, hormone, hormone analog, protein, peptide or metal ion.

18) The method of claim 17, wherein said small molecule is quinic acid.

19) The method of claim 15, wherein said transcription factor is QA-1F (‘QF’).

20) The method of claim 15, wherein said DNA sequence is QUAS.

21) The method of claim 15, wherein said repressor of said transcription factor is QA-1S (‘QS’).

22) A method for lineage tracing in an organism marking any two siblings and progeny from a single cell division, the method comprising
a) a first binary expression system comprising regulatory genes from Neurospora crassa;
b) a second binary expression system;
c) a recombination site located proximally to a GAL80 transgene on any chromosome arm;
d) a recombination site located on a homologous chromosome and proximally to a QA-1S transgene;
e) one or more effector transgenes;
f) a recombinase transgene for recombining said recombination sites c) and d).

23) The method of claim 22, wherein said second binary expression system is GAL4/UAS.

24) A method for genetic mosaic analysis in an organism, the method comprising
a) an inducible binary expression system comprising regulatory genes from Neurospora crassa;
b) a mutation in a gene;
c) a recombination site located proximally to said mutation;
d) a recombination site on a homologous chromosome;
e) a QA-1S transgene located distally from said recombination site d);
f) one or more effector transgenes;
g) a recombinase transgene for recombining said recombination sites c) and d).
